SEQUENCE LISTING (12 sequences)

SEQ ID NO: 1 - protein, 239 aa, Homo sapiens
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - protein, 475 aa, Homo sapiens [sequence omitted]

SEQ ID NO: 3 - protein, 177 aa, Homo sapiens
MEALILEPSLYTVKAILILDNDGDRLFAKYYDDTYPSVKEQKAFEKNIFNKTHRTDSEIALLEGLTVVYKSSIDLYFYVIGSSYENELMLMAVLNCLFDSLSQMLRKNVEKRALLENMEGLFLAVDEIVDGGVILESDPQQVVHRVALRGEDVPLTEQTVSQVLQSAKEQIKWSLLR

SEQ ID NO: 4 - protein, 531 aa, Homo sapiens
MDGIVPDIAVGTKRGSDELFSTCVTNGPFIMSSNSASAANGNDSKKFKGDSRSAGVPSRVIHIRKLPIDVTEGEVISLGLPFGKVTNLLMLKGKNQAFIEMNTEEAANTMVNYYTSVTPVLRGQPIYIQFSNHKELKTDSSPNQARAQAALQAVNSVQSGNLALAASAAAVDAGMAMAGQSPVLRIIVENLFYPVTLDVLHQIFSKFGTVLKIITFTKNNQFQALLQYADPVSAQHAKLSLDGQNIYNACCTLRIDFSKLTSLNVKYNNDKSRDYTRPDLPSGDSQPSLDQTMAAAFGLSVPNVHGALAPLAIPSAAAAAAAAGRIAIPGLAGAGNSVLLVSNLNPERVTPQSLFILFGVYGDVQRVKILFNKKENALVQMADGNQAQLAMSHLNGHKLHGKPIRITLSKHQNVQLPREGQEDQGLTKDYGNSPLHRFKKPGSKNFQNIFPPSATLHLSNIPPSVSEEDLKVLFSSNGGVVKGFKFFQKDRKMALIQMGSVEEAVQALIDLHNHDLGENHHLRVSFSKSTI

SEQ ID NO: 5 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 6 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 7 - protein, 1255 aa, Homo sapiens [sequence omitted]

SEQ ID NO: 8 - protein, 1210 aa, Homo sapiens [sequence omitted]

SEQ ID NO: 9 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 10 - protein, 435 aa, Homo sapiens
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNGKCREFFPNHQWVKEETQEDKDCPIKEEKGSPLNAAPYGIESMSQDTEVRSRVVGGSLRGAQAASPAKGEPSLPEKDEDHALSYWKPFLVNMCVATVLTAGAYLCYRFLFNSNT

SEQ ID NO: 11 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 12 - protein, 889 aa, Homo sapiens
MEEGAPRQPGPSQWPPEDEKEVIRRAIQKELKIKEGVENLRRVATDRRHLGHVQQLLRSSNRRLEQLHGELRELHARILLPGPGPGPAEPVASGPRPWAEQLRARHLEALRRQLHVELKVKQGAENMTHTCASGTPKERKLLAAAQQMLRDSQLKVALLRMKISSLEASGSPEPGPELLAEELQHRLHVEAAVAEGAKNVVKLLSSRRTQDRKALAEAQAQLQESSQKLDLLRLALEQLLEQLPPAHPLRSRVTRELRAAVPGYPQPSGTPVKPTALTGTLQVRLLGCEQLLTAVPGRSPAAALASSPSEGWLRTKAKHQRGRGELASEVLAVLKVDNRVVGQTGWGQVAEQSWDQTFVIPLERARELEIGVHWRDWRQLCGVAFLRLEDFLDNACHQLSLSLVPQGLLFAQVTFCDPVIERRPRLQRQERIFSKRRGQDFLRASQMNLGMAAWGRLVMNLLPPCSSPSTISPPKGCPRTPTTLREASDPATPSNFLPKKTPLGEEMTPPPKPPRLYLPQEPTSEETPRTKRPHMEPRTRRGPSPPASPTRKPPRLQDFRCLAVLGRGHFGKVLLVQFKGTGKYYAIKALKKQEVLSRDEIESLYCEKRILEAVGCTGHPFLLSLLACFQTSSHACFVTEFVPGGDLMMQIHEDVFPEPQARFYVACVVLGLQFLHEKKIIYRDLKLDNLLLDAQGFLKIADFGLCKEGIGFGDRTSTFCGTPEFLAPEVLTQEAYTRAVDWWGLGVLLYEMLVGECPFPGDTEEEVFDCIVNMDAPYPGFLSVQGLEFIQKLLQKCPEKRLGAGEQDAEEIKVQPFFRTTNWQALLARTIQPPFVPTLCGPADLRYFEGEFTGLPPALTPPAPHSLLTARQQAAFRDFDFVSERFLEP

(19) United States
(12) Patent Application Publication
(10) Pub. No.: US 2021/0403644 A1
(43) Pub. Date: Dec. 30, 2021

(54) Method For The Degradation Of Endogenous Protein

(71) Applicant: University of South Carolina, Columbia, SC (US)

(72) Inventor: Peisheng Xu, Chapin, SC (US)

(73) Assignee: University of South Carolina, Columbia, SC (US)

(21) Appl. No.: 17/242,647

(22) Filed: Apr. 28, 2021

Related U.S. Application Data
(60) Provisional application No. 63/045,275, filed on Jun. 29, 2020.

(51) Int. Cl. C08G 69/48; C08G 69/26; C08G 69/10; A61K 9/51; A61K 47/64

(52) U.S. Cl. CPC C08G 69/48; C08G 69/26; A61K 47/64; A61K 9/5146; C08G 69/10

(57) ABSTRACT

Described herein are systems and methods for the degradation of endogenous protein with the help of a nanocarrier, which has the advantage of easy scale-up and feasibility for in vivo application.

### STATEMENT REGARDING FEDERALLY SPONSORED RESEARCH

This disclosure was made with government support under R01 AG054839 awarded by the National Institute of Health. The government may have certain rights in the invention.

### TECHNICAL FIELD

The present disclosure provides systems and methods for the degradation of endogenous protein with the help of a nanocarrier, which has the advantage of easy scale-up and feasibility for in vivo application.

### BACKGROUND

The currently available tools for degrading an endogenous protein are not specific and/or can cause permanent genetic modification. The traditional endogenous protein degradation method, Trim-Away, requires microinjection or an electroporation step, which is not safe and convenient for large scale and in vivo applications.

Accordingly, it is an object of the present invention to provide a an intracellular antibody delivery method to effectively and specifically degrade an endogenous protein for large scale and future in vivo application.

### SUMMARY

The above objectives are accomplished according to the present disclosure by providing a method for creating an intracellular antibody delivery device. The method may include forming a polymer for introduction to a cell, producing a polymeric nanogel via crosslinking, introducing an antibody or protein to the polymeric nanogel, wherein the antibody or protein is internalized by the nanogel, uptake by at least one cell of the polymeric nanogel, and cleaving a self-immolative linker present in the polymeric nanogel to release the antibody or protein within the at least one cell. Further, the polymer may include PDA-PEG-NPC. Again, the polymer may be PDA-PEG-BSA-Cy5 or PDA-PEG-Cy3. Still, the PDA-PEG-NPC polymer may include p-nitrophenylcarbonate (NPC) moieties in side chains. Again further, the NPC moieties may be replaced by lysine groups of the antibody or protein to produce antibody or protein conjugated polymers. Yet still, the nanogel may include PBS buffer, TCEP and ethylenediamine and deionized water. Still further, the nanogel may be modified with RGD peptide. Yet again, the at least one cell may be a human breast cancer cell. Still further, the method may degrade a protein containing SEQ ID NO: 3 in the at least one human breast cancer cell.

In an alternative embodiment, the current disclosure may provide a method for degrading intracellular proteins in at least one TRIM21 expressing cell. The method may include forming at least one protein loaded nanogel wherein the protein loaded nanogel may comprise at least one polymer nanogel and at least one protein and the at least one protein comprises at least one antibody, at least one nanobody, or a combinations of at least one antibody and at least one nanobody. Further, the TRIM21 expressing cell may be a naturally occurring TRIM21 expressing cell or a cell with acquired TRIM21 expression. Still, the at least one antibody may comprise anti COPZ1 antibody, anti PTBP1 antibody, anti PD-L1 antibody, anti PD-1 antibody, anti-Her2 antibody, anti EGFR antibody, anti survivin antibody, anti PTP1B antibody, anti VEGF antibody, anti PKN3 antibody. Yet further, the at least one nanobody may comprise anti COPZ1 nanobody, anti PTBP1 nanobody, anti PD-L1 nanobody, anti PD-1 nanobody, anti-Her2 nanobody, anti EGFR nanobody, anti survivin nanobody, anti PTP1B nanobody, anti VEGF nanobody, anti PKN3 nanobody. Still further, the protein-laded nanogel system may be used for treating cancer, Alzheimer's diseases, Parkinson's disease, multiple sclerosis, neonatal hypoxic-ischemic, stroke, Amyotrophic lateral sclerosis, Huntington's disease, spinal cord injury, brain injury, retina injury, post-traumatic stress disorder, and frontotemporal dementia, and/or traumatic brain injury.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows a schematic illustration of: A) the Trim-Away technique; B) the fabrication of polymer nanogels using protein/antibody-conjugated polymers; and C) the mechanism of traceless release of protein/antibody from the loaded nanogels in the presence of reducing agent GSH.

FIG. 2 shows at: A) Gel electrophoresis of (1) protein marker, (2) free BSA, (3) polymer PDA-PEG-BSA, and (4) GSH-treated PDA-PEG-BSA; B) Fluorescence emission spectra of a mixture of nanogels NG-BSA-Cy5 and NG-Cy3, and nanogel NG-BSA-Cy5-Cy3 in the absence and presence of GSH (λex=525 nm); and C) Confocal fluorescence images of MCF-7 cells incubated with NG-BSA-Cy5-Cy3 at different incubation times.

FIG. 3 shows at: A) Size distribution and B) TEM image of nanogel NG-aGFP; C) Gel electrophoresis of (1) free anti-GFP antibody, (2) nanogel NG-aGFP, (3) GSH-treated NG-aGFP, and (4) protein marker; D) Fluorescence images and E) relative fluorescence intensity data of TRIM21-transfected MCF-7/GFP cells after incubation with non-loading empty nanogel (NG-empty), free anti-GFP antibody, and NG-aGFP nanogel; F) Relative fluorescence intensity of TRIM21-transfected MCF-7/GFP cells after incubation with free anti-GFP and relevant nanogels equivalent to varied concentrations of anti-GFP; G) Fluorescence images of TRIM21-transfected MCF-7/GFP cells, GFP (green) and TRIM21 (red) channels, after incubation with free anti-GFP and nanogels at an anti-GFP equivalent concentration of 20 μg/mL for 9 h; and H) Relative fluorescence intensity of TRIM21-transfected MCF-7/GFP cells after incubation with NG-aGFP and NG-aGFP-R equivalent to 20 μg/mL anti-GFP.

FIG. 4 shows at: A) Size distribution and B) TEM image of nanogel NG-aCOPZ1-R; C) Cell viability of TRIM21-transfected MCF-7 cells after incubation with free anti-COPZ1 antibody, NG-empty, NG-aCOPZ1, and NG-aCOPZ1-R nanogels at varied anti-COPZ1 equivalent concentrations; D) The COPZ1 protein expression in TRIM21-transfected MCF-7 cells measured by western blotting assay after incubation with free anti-COPZ1 and relevant nanogels; E) the corresponding quantitative analysis; and F) Fluorescence of GFP (green) and TRIM21 (red) emitted from TRIM21-transfected MCF-7/GFP cells before and after Trim-Away with NG-aCOPZ1-R nanogel.

FIG. 5 shows synthesis of protein/antibody conjugated polymers.

FIG. 6 shows ¹H NMR spectrum of PDA-PEG-NPC.

FIG. 7 shows size distribution of nanogel NG-BSA.

FIG. 8 shows Zeta potential of nanogels NG-BSA, NG-aGFP, NG-aGFP-R, NG-aCOPZ1, and NG-aCOPZ1-R.

FIG. 9 shows a TEM image of nanogel NG-BSA.

FIG. 10 shows absorption and fluorescent emission spectra of BSA-Cy5.

FIG. 11 shows fluorescence of GFP (green) and TRIM21 (red) emitted from MCF-7/GFP cells before and after transfection with pmCherry-C1-mTRIM21 plasmid.

FIG. 12 shows fluorescence images of MCF-7/GFP cells without TRIM21-transfection after incubation with NG-empty, free anti-GFP, and NG-aGFP nanogel at an anti-GFP equivalent concentration of 100 μg/mL for 6 h.

FIG. 13 shows relative fluorescence intensity of MCF-7/GFP cells without TRIM21-transfection after incubation with NG-empty, free anti-GFP, and NG-aGFP nanogel at an anti-GFP equivalent concentration of 100 μg/mL for 6 h.

FIG. 14 shows size distribution of nanogel NG-aGFP-R.

FIG. 15 relative intensity of both GFP and TRIM21 fluorescence in TRIM21-transfected MCF-7/GFP cells after incubation with free anti-GFP and nanogels at an anti-GFP equivalent concentration of 20 μg/mL for 9 h.

FIG. 16 shows cell viability of TRIM21-transfected MCF-7/GFP cells after incubation with NG-empty, free anti-GFP, NG-aGFP, and NG-aGFP-R nanogels at varying anti-GFP equivalent concentrations.

FIG. 17 shows size distribution of nanogel NG-aCOPZ1.

FIG. 18 shows cell viability of MCF-7 cells after incubation with NG-empty, free anti-COPZ1, NG-aCOPZ1, and NG-aCOPZ1-R nanogels at varying anti-COPZ1 equivalent concentrations.

FIG. 19 shows cell viability of TRIM21-transfected NIH-3T3 cells after incubation with free anti-COPZ1, NG-empty, NG-aCOPZ1, and NG-aCOPZ1-R at varying anti-COPZ1 equivalent concentrations.

The figures herein are for illustrative purposes only and are not necessarily drawn to scale.

### DETAILED DESCRIPTION OF A PREFERRED EMBODIMENT

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intraderm al, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, sub arachnoid, subconjunctival, subcutaneous, sublingual, submucosal, topical, transderm al, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein “cancer” can refer to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basa cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “chemotherapeutic agent” or “chemotherapeutic” refers to a therapeutic agent utilized to prevent or treat cancer.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of a pharmaceutical formulation thereof calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g. the monomers are biologically important (e.g. an amino acid), natural, or synthetic.

As used herein, the term “radiation sensitizer” refers to agents that can selectively enhance the cell killing from irradiation in a desired cell population, such as tumor cells, while exhibiting no single agent toxicity on tumor or normal cells.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

The current disclosure provides a tool for specific protein degradation in molecular biology, as well as nanomedicine for treatment of maladies such as cancer and Alzheimer's disease.

The currently available tools for degrading an endogenous protein are not specific or can cause permanent genetic modification. The traditional endogenous protein degradation method, Trim-Away, requires microinjection or an electroporation step, which is not safe and convenient for large scale and in vivo applications. The current disclosure provides an intracellular antibody delivery method to effectively and specifically degrade an endogenous protein for large scale and future in vivo application.

Proteins play crucial roles in the human body, including enzymes, carriers, structure building blocks, hormone signaling, defense, and storage. The malfunction of proteins causes various diseases, such as Alzheimer's disease, amyotrophic lateral sclerosis, cystic fibrosis, type 2 diabetes, and cancer. Antibodies, a functional member of the protein family that can bind to proteins with specificity and high affinity, are an ideal candidate for protein therapy, as they can be produced for any protein using the well-established phage display or hybridoma technology.

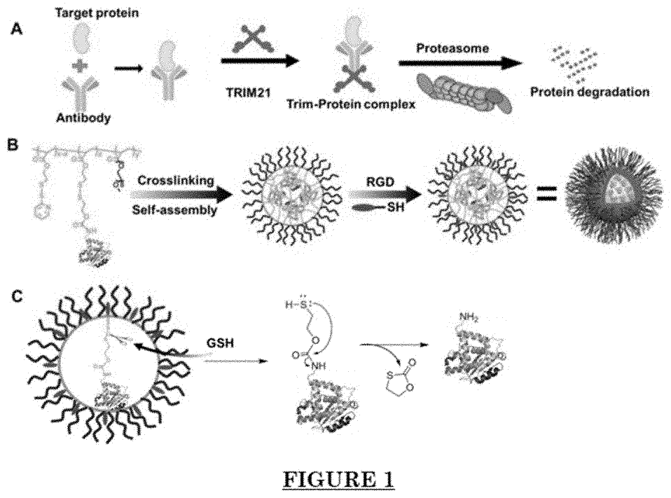

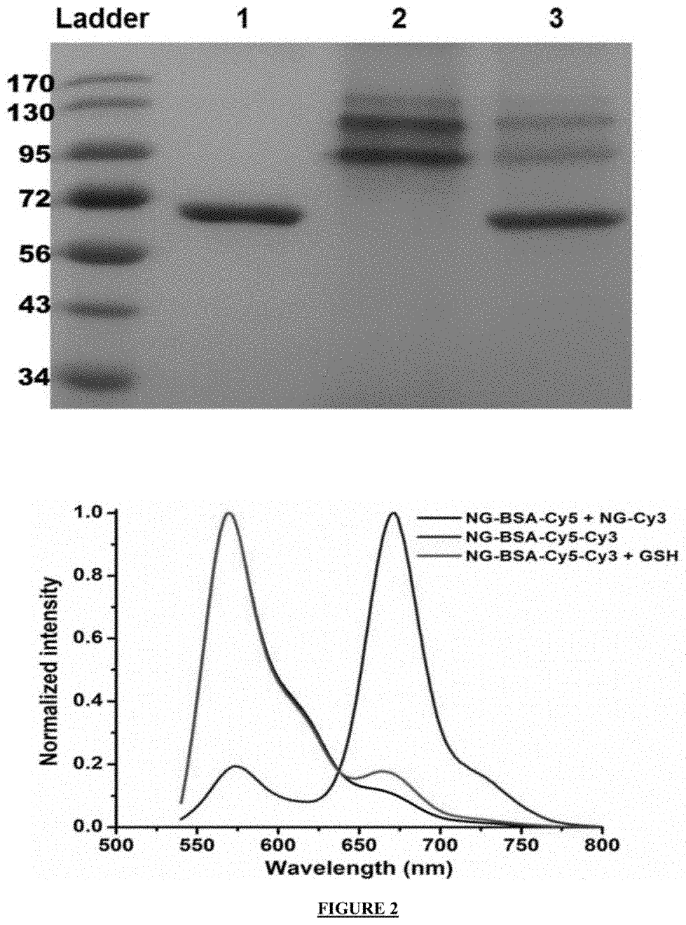

Clift et al. discovered a so-called “Trim-Away” method, which utilizes antibodies to degrade endogenous proteins in mammalian cells without prior modification of the genome or mRNA. The mechanism of Trim-Away involves the intracellular antibody receptor TRIM21, which is an E3 ubiquitin ligase that binds to the Fc domain of antibodies, and TRIM21 is commonly expressed in various cell types because of its indispensable physiological role. As illustrated in FIG. 1 at A, the target protein is bound by the antibody, followed by TRIM21-mediated ubiquitination to generate a protein complex, which turns into a proteasome that subsequently degrades. Trim-Away is a highly efficient technique to degrade target proteins in cells, and it can be readily applied to a broad spectrum of intracellular proteins. However, the extensive application of Trim-Away is severely hindered by the cell membrane impermeability of antibodies. Clift et al. delivered antibodies into cells by microinjection and electroporation techniques, which both bring damages to cells, and their in vivo applications are limited.

With the rapid development of diverse drug delivery systems, nanoparticulate delivery vehicles designed for intracellular delivery of protein/antibody surged in the past decade, including inorganic nanoparticles, liposomes, and polymeric nanocarriers. Our group has investigated drug-loaded polymeric micelles and nanogels for cancer and central nervous system diseases. Herein, we intend to develop a safer and more convenient version of Trim-Away through employing polymer nanogels as the antibody delivery approach instead of microinjection and electroporation. Based on that, we attempt to utilize this new technique to degrade a vital intracellular protein of cancer cells and thereby to kill the cells.

Materials

2-Mercaptoethanol (BME), glacial acetic acid, ethylenediamine, L-glutathione (GSH), bovine serum albumin (BSA), and phosphate buffered saline (PBS) were purchased from Sigma-Aldrich Chemical Co. (St. Louis, Mo., USA). Triethylamine (TEA), pyridine, tris(2-carboxyethyl)phosphine (TCEP), 4-nitrophenyl chloroformate (NPC), and (3-(4,5-dimethylthiazol-2-yl)-2,5-diphenyltetrazolium bromide (MTT) were purchased from Tokyo Chemical Industry Co., Ltd (Portland, Oreg., USA). Anti-GFP antibody was purchased from Abcam PLC. (Cambridge, Mass., USA). Anti-COPZ1 antibody was purchased from Santa Cruz Biotechnology, Inc. (Santa Cruz, Calif., USA). Plasmid pmCherry-C1-mTrim21, originally created by Schuh et al., was purchased from Addgene (Watertown, Mass., USA). Cyanine3 NHS ester (Cy3-NHS) and Cyanine5 NHS ester (Cy5-NHS) were purchased from Lumiprobe Co. (Cockeysville, Md., USA). Cyclic Arg-Gly-Asp-D-Phe-Cys peptide (RGD) was purchased from GL Biochem (Shanghai) Ltd. (Shanghai, China). Gibco™ Dulbecco's modified Eagle's medium (DMEM), fetal bovine serum (FBS), penicillin-streptomycin (PS), trypsin-EDTA, Pierce™ BCA Protein Assay Kit, Lipofectamine® 3000 Transfection Kit, anti-β-actin antibody, and Invitrogen™ Hoechst 33342 were purchased from Thermo Fisher Scientific, Inc. (Waltham, Mass., USA). Deuterated solvents were purchased from Cambridge Isotope Laboratories, Inc. (Andover, Mass., USA). All the other solvents used in this research were purchased from Sigma-Aldrich Chemical Co. (St. Louis, Mo., USA) and directly used without further purification.

Polymers PDA-PEG-BME and PDA-PEG-Cy3 were prepared as described herein.

Synthesis of PDA-PEG-NPC

30 mg PDA-PEG-BME and 7.2 mg 4-nitrophenyl chloroformate (NPC) were dissolved in 500 μL dichloromethane (DCM). After cooled in ice bath for 30 min, 20 μL pyridine was added dropwise, and the reaction solution was stirred for 24 h at room temperature in the dark. The produced polymer was purified through dialysis of the reaction mixture towards DMSO using Spectra/Pore dialysis tube (MWCO: 8 kDa). The desired product PDA-PEG-NPC was collected through precipitation with ice-cold diethyl ether. Further removal of DMSO residue was performed twice via DCM/ice-cold diethyl ether precipitation. After in vacuo dryness in the dark for 48 h (28 mg, 87.5%), the polymer was analyzed by NMR to confirm its chemical structure.

Preparation of Nanogels

To a pre-cooled solution of 50 mg polymer PDA-PEG-NPC in 5 mL PBS buffer (pH 8.5), 1 mg protein/antibody dissolved in 1 mL PBS buffer was added dropwise at 4° C. under vigorous stirring, and the resulted solution was stirred for 48 h at 4° C. in the dark. The process of reaction was monitored by measuring the absorbance of released side product 4-nitrophenol at 400 nm using UV-Vis spectroscopy as reported in literature. When the reaction was completed, 2.6 mg TCEP and 1.1 mg ethylenediamine dissolved in 0.2 mL pre-cooled deionized water was added for crosslinking and the solution was stirred for 24 h at 4° C. Then the produced nanogels were purified through dialysis in Spectra/Pore dialysis tube (MWCO: 100 kDa) against PBS buffer for 48 h at 4° C. The final nanogels were stored in PBS (pH 7.4) at 4° C. for use. For the nanogels modified with Cy3, polymer PDA-PEG-Cy3 was mixed into the reaction solution before the crosslinking step. The concentration of Cy3 was measured by microplate reader (λex=555 nm, λgem=570 nm). For the nanogels post-decorated with RGD peptide, the nanogel dispersion in PBS buffer (pH 7.4) was added RGD solution (1 mg/mL) in PBS buffer and then stirred overnight at 4° C. The RGD-modified nanogels were purified through dialysis in Spectra/Pore dialysis tube (MWCO: 8 kDa) against PBS buffer for 48 h at 4° C. The particle size, size distribution, and zeta potential of the nanogels were determined by dynamic light scattering (DLS), recorded on Zetasizer (Zetasizer Nano ZS, Malvern Instruments Ltd, Malvern, UK). The physical morphology was observed using Hitachi HT7800 transmission electron microscopy (TEM, Hitachi High-Technologies Corporation, Tokyo, Japan).

The amount of protein contained in nanogels was analyzed using the Pierce™ BCA Protein Assay Kit, following the reported method. The loading content (LC) and loading efficiency (LE) of Protein/antibody were calculated by the following equations.

$$LC ( \% ) = \frac{mass of protein in nanogel}{mass of nanogel} \times 100 \%$$

$$LE ( \% ) = \frac{mass of protein in nanogel}{mass of protein fed} \times 100 \%$$

Protein/Antibody Release

The release of protein/antibody from the nanogels was analyzed with SDS-PAGE gel electrophoresis. 12 μL of different samples were mixed with 10 μL loading buffer, and 20 μL of each sample was loaded onto gel. For the redox-responsive release test, the nanogels were treated with 0.1 M GSH for 12 h prior to gel electrophoresis.

Fluorescence Labeling of Protein

For cellular uptake studies of protein/antibody and nanogels, bovine serum albumin (BSA) was labeled with fluorescent dye Cyanine5 (Cy5) to form BSA-Cy5. In brief, 5 mg of BSA dissolved in 1 mL NaHCO₃ buffer (0.1 M, pH=8.5) was cooled in ice bath for 30 min, and then 50 μL freshly prepared Cy5-NHS solution (10 mg/mL in DMSO) was added dropwise in the dark. The reaction mixture was protected from light and stirred at room temperature overnight. The produced BSA-Cy5 was purified by size exclusion chromatography using Sephadex G-25 (GE Healthcare).

Cell Culture

Human breast cancer MCF-7 cells, green fluorescence protein (GFP) expressed MCF-7/GFP cells, and mouse embryonic fibroblast NIH-3T3 cells were cultured in Gibco™ DMEM supplemented with 10% FBS, 100 units/mL penicillin, and 100 μg/mL streptomycin at 37° C. in 75 mL culture flasks under a humidified atmosphere of 5% CO₂. Cells were sub-cultured when the cell confluence reached ˜80%.

Cellular Internalization

The cellular internalization of nanogels was examined by confocal fluorescence microscopy in MCF-7 cells. Cells were seeded in 35 mm² Petri dish with a glass window at a density of 200,000 cells per dish for 24 h. Then cells were incubated with Cy3 and BSA-Cy5 co-loaded nanogel NG-BSA-Cy5-Cy3 (5 μg/mL for Cy5, 4.1 μg/mL for Cy3) for 3 h. Cells without any incubation were utilized as negative control. In the positive control group, cells were co-incubated with nanogels NG-Cy3 and NG-BSA-Cy5. Then the medium was replaced with fresh medium and cells were fixed with 4% paraformaldehyde in PBS for 10 min at predetermined time points. After removal of paraformaldehyde, the nuclei of cells were stained with Hoechst 33342 for 10 min, and then cells were imaged under a confocal fluorescence microscope (LSM 700, Carl-Zeiss Inc.).

Cell Viability Assay

The cytotoxicity of free antibody and nanogels was evaluated by MTT assay. Cells were seeded in 96-well plates at a density of 5,000 cells per well for 24 h at 37° C. with 5% CO₂. Then cells were incubated with free antibody and nanogels in fresh medium for 48 h. In the control group, cells were allowed to grow without any treatment. After that, the medium was replaced with fresh medium containing MTT reagent (final concentration 1 mg/mL) and cells were further incubated for 4 h. The purple MTT crystal was dissolved with MTT stop solution and the optical density at 595 nm was recorded on microplate reader (SpectraMax i3x, Molecular Devices, LLC.).

Protein Expression

For the expression of protein Trim21, cells were transfected with pmCherry-C1-mTrim21 plasmid as reported in literature. Plasmids were transfected using Lipofectamine® 3000 Transfection Kit according to manufacturer's instructions 12 h prior to Trim-Away assay. Successful transfection was indicated by the observed cherry fluorescence emitted from cells.

Trim-Away Assay

The Trim-Away of protein GFP was conducted in TRIM21-transfected MCF-7/GFP cells. For fluorescence imaging, cells seeded in 35 mm² Petri dish with a glass window were incubated with free anti-GFP antibody and anti-GFP loaded nanogels. Cells with no incubation were utilized as control. At predetermined post-incubation time points, cells were imaged by fluorescence microscopy. To quantitatively analyze the Trim-Away efficiency, the fluorescence of cells seeded in 96-well plates after incubation with anti-GFP and nanogels was measured by microplate reader at different time points (λex=395 nm, λgem=505 nm).

For the Trim-Away of protein COPZ1, TRIM21-transfected cells were incubated with free anti-COPZ1 antibody and anti-COPZ1 loaded nanogels at varied concentrations for 48 h. Cells in the control group were allowed to grow with no incubation. Then the medium was replaced with fresh medium containing MTT reagent (final concentration 1 mg/mL) and cells were further incubated for 4 h. The purple MTT crystal was dissolved with MTT stop solution and the optical density at 595 nm was recorded by microplate reader.

Western Blotting Assay

TRIM21-transfected MCF-7 cells were incubated with free anti-COPZ1, NG-aCOPZ1 and NG-aCOPZ1-R nanogels (equivalent to 40 μg/mL anti-COPZ1) for 10 h and then lysed for 30 min at 4° C. The protein concentration of each sample was measured by Bradford assay. The cell lysates were separated by SDS-PAGE and transferred to a PVDF membrane, which was activated by methanol for 30 min prior to use. The transferred membrane was blocked for 1.5 h at room temperature with 5% BSA, washed with Tris-buffered saline with Tween-20 (TBST buffer), and incubated overnight at 4° C. with anti-COPZ1 (1:500) or anti-β-actin (1:1000). Chemiluminescence detection was performed with the corresponding second antibody conjugated with HRP. Images were acquired using ChemiDoc™ Touch Imaging System (Bio-Rad Laboratories, Inc.).

Results

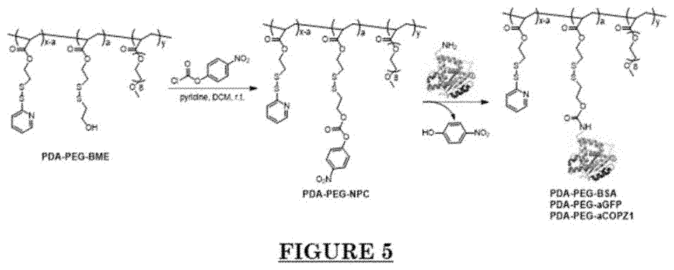

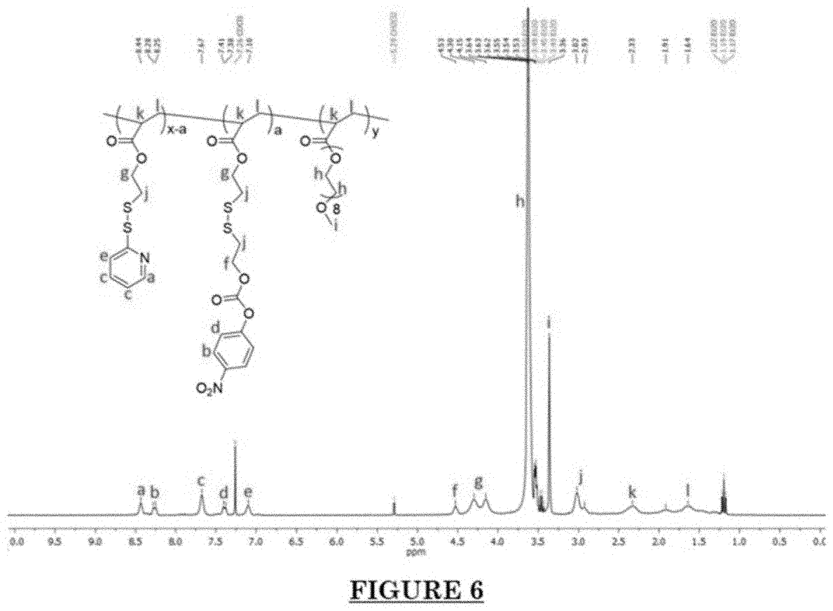

A polymer PDA-PEG-NPC bearing p-nitrophenylcarbonate (NPC) moieties in side chains was synthesized by attaching NPC to polymer PDA-PEG-BME, see FIG. 5, which was prepared as we previously reported, and characterized by nuclear magnetic resonance spectroscopy, see FIG. 6. The protein/antibody was reacted with PDA-PEG-NPC to produce protein/antibody conjugated polymers, in which the NPCs are replaced by the reactive lysine groups of the protein/antibody, see FIG. 5. The redox-sensitive linker between the protein/antibody and the polymer backbone is self-immolative and the biodegradable disulfide bond can be readily cleaved by reducing agents such as glutathione (GSH). As illustrated in FIG. 1 at B, the resulted polymers were fabricated into polymer nanogels via the crosslinking reaction induced by tris(2-carboxyethyl)phosphine (TCEP). The polymer nanogels were further modified with a tumor-targeting ligand RGD (cyclic Arg-Gly-Asp-D-Phe-Cys peptide). After being taken up by cells, the self-immolative linker would be cleaved by intracellular GSH, leading to the traceless release of the protein/antibody, see FIG. 1 at C, which makes the nanogels more advantageous for delivering protein/antibody into cancer cells since the intracellular level of GSH in cancer cells (2-10 mM) is much higher than that in normal cells and extracellular matrices (2-20 μM).

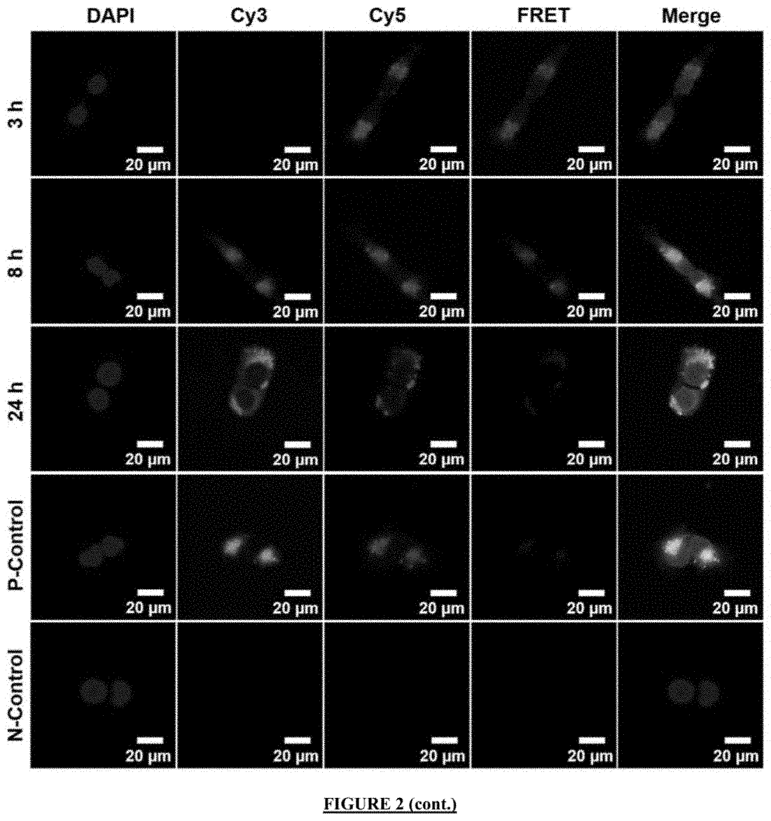

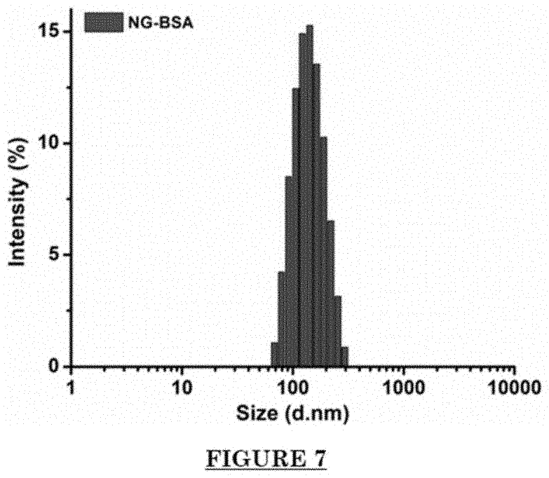

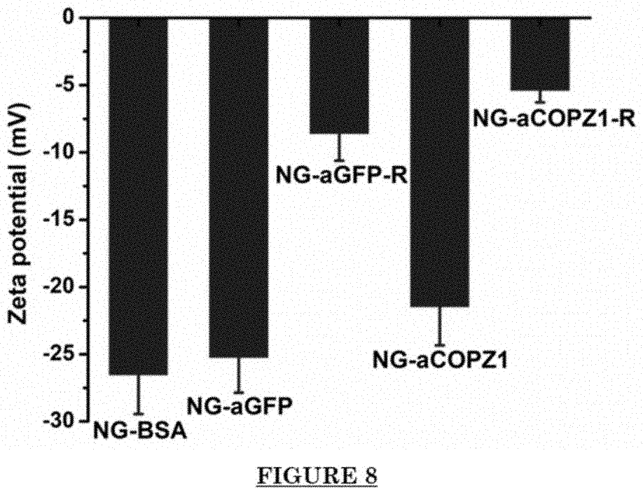

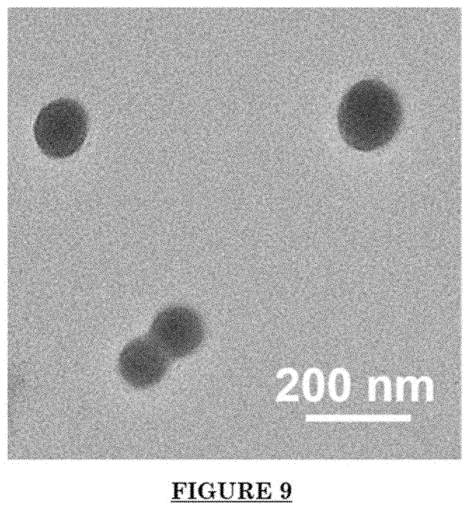

To verify our design strategy, bovine serum albumin (BSA) was utilized as a protein model to investigate the conjugation and subsequent release of protein/antibody from the polymer by gel electrophoresis using SDS-PAGE gel. As displayed in FIG. 2 at A, the protein band of free BSA disappeared in the lane loaded with polymer PDA-PEG-BSA, which indicates the success of protein conjugation. However, in the lane loaded with GSH-pretreated PDA-PEG-BSA, a clear band of BSA was observed, suggesting the redox-responsive release of the protein. Then polymer PDA-PEG-BSA was cross-linked by TCEP to fabricate polymeric nanogel NG-BSA, and the unreacted NPCs were eliminated by ethylenediamine. The amount of BSA contained in NG-BSA was determined using BCA protein assay kit according to the reported method. The loading content (LC) and loading efficiency (LE) of BSA were calculated to be 3.4% and 77%, respectively. Dynamic light scattering (DLS) determined that NG-BSA had a hydrodynamic size of 135.4 nm with a dispersity of 0.12, see FIG. 7, and a negative zeta potential, see FIG. 8. Transmission electron microscopy (TEM) showed that NG-BSA had a spherical shape and a diameter of 109.3 nm in dry state, see FIG. 9.

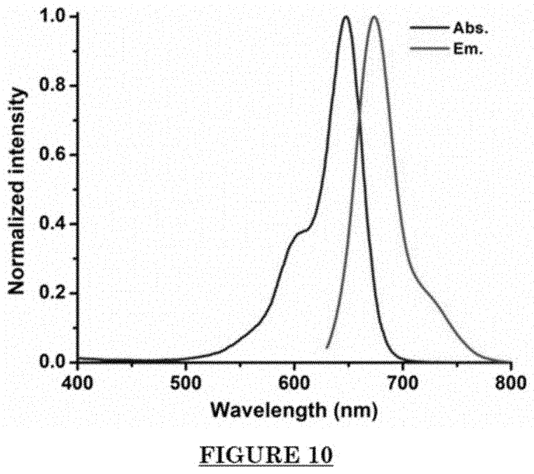

In order to study the cellular uptake and intracellular release of the protein from the nanogel using fluorescence resonance energy transfer (FRET) technique, we labelled BSA with a fluorescent dye Cyanine5 (Cy5) to generate BSA-Cy5. The absorption and fluorescent emission wavelength of BSA-Cy5 was determined to be 648 nm and 670 nm, respectively, see FIG. 10. Polymer PDA-PEG-BSA-Cy5 was synthesized in the same way as PDA-PEG-BSA using BSA-Cy5 instead of BSA. Then it was mixed with a Cyanine3 (Cy3, λ_{abs}=555 nm, λₑₘ=570 nm) labelled polymer PDA-PEG-Cy3, which was prepared as we previously reported, to fabricate a Cy5 and Cy3 co-labelled nanogel NG-BSA-Cy5-Cy3, since the fluorescent dyes Cy3 and Cy5 make an efficient FRET pair. As shown in FIG. 2 at B, when excited at 525 nm, a wavelength that can excite fluorophore Cy3 and spares Cy5, intense fluorescence of Cy3 (568 nm) and negligible fluorescence of Cy5 (670 nm) were observed from a mixture of nanogels NG-BSA-Cy5 and NG-Cy3, which were made from polymers PDA-PEG-BSA-Cy5 and PDA-PEG-Cy3, respectively. On the contrary, nanogel NG-BSA-Cy5-Cy3 emitted weak fluorescence of Cy3 and very strong fluorescence of Cy5 under the same conditions, which demonstrates that efficient FRET phenomenon occurred between the Cy3 and Cy5 fluorophores of NG-BSA-Cy5-Cy3. However, in the presence of GSH, the fluorescence of Cy3 emitted by NG-BSA-Cy5-Cy3 was significantly enhanced and that of Cy5 decreased simultaneously, which is quite similar to the fluorescence emission pattern of the mixed nanogels NG-BSA-Cy5 and NG-Cy3. These observations indicate that BSA-Cy5 was released from the nanogel by GSH so that the two fluorophores were no long in proximity and thus the FRET phenomenon was inhibited.

To determine whether the GSH-induced FRET-inhibition phenomenon could occur inside cells, we incubated human breast cancer MCF-7 cells with nanogel NG-BSA-Cy5-Cy3 and recorded the fluorescence emitted from cells using confocal fluorescence microscopy. First of all, as shown in FIG. 2 at C, the strong fluorescence of cells confirmed that the protein loaded nanogels could be efficiently taken up by cells. Moreover, obvious FRET phenomenon was observed after 3 h incubation with NG-BSA-Cy5-Cy3, as evidenced by the very weak fluorescence (green) of Cy3 and strong fluorescence (red) of Cy5. At 8 h post-incubation, the significantly increased fluorescence of Cy3 and declined fluorescence of Cy5 indicated that some of the Cy5-labelled protein BSA-Cy5 was liberated and the FRET efficiency decreased accordingly. After 24 h incubation, the FRET phenomenon nearly disappeared and the fluorescence of cells was almost the same as that of cells co-incubated with nanogels NG-BSA-Cy5 and NG-Cy3 in the positive control group, which suggest that most of the loaded BSA-Cy5 had already been released from NG-BSA-Cy5-Cy3. All the results confirm the effectiveness of our design strategy that the polymer nanogel can deliver the loaded protein into cells and release it on demand.

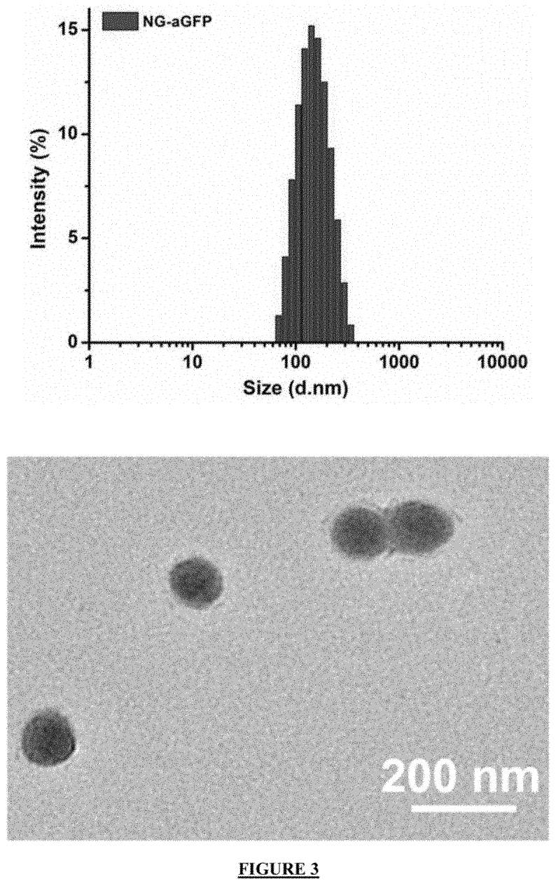

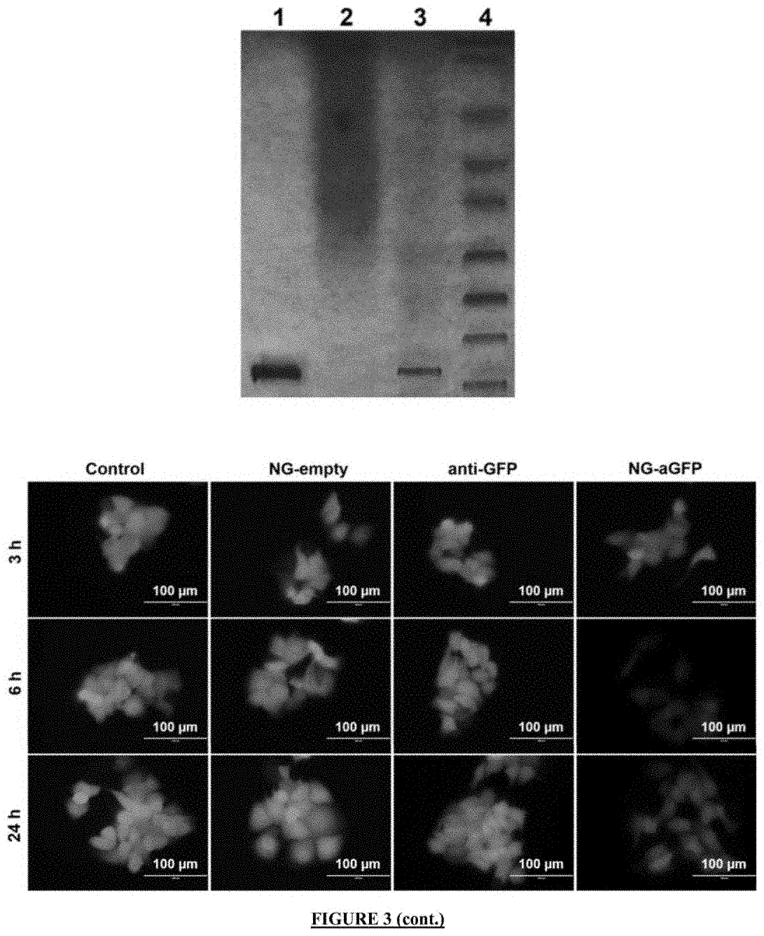

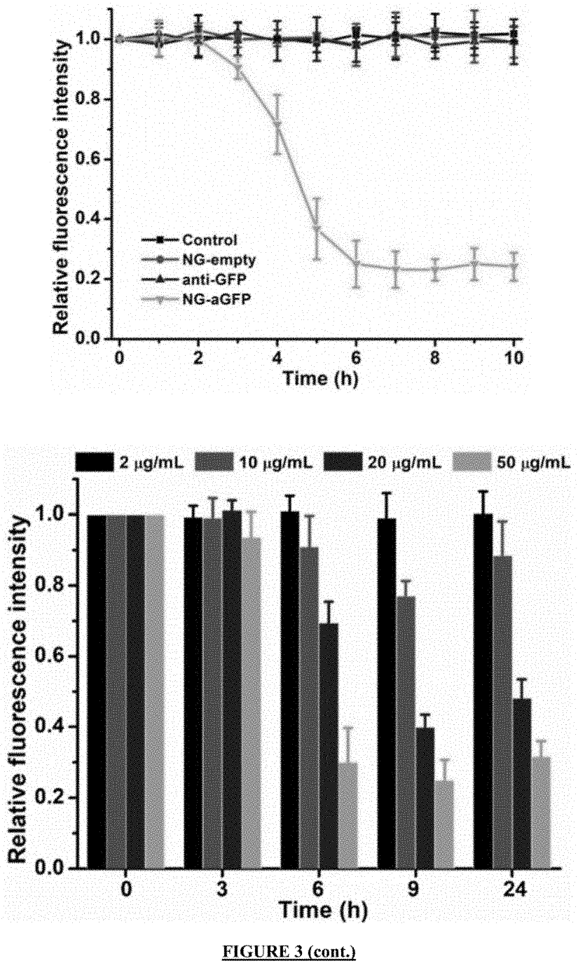

Then anti-GFP, an antibody for green fluorescence protein (GFP), was conjugated to the polymer by reacting anti-GFP with polymer PDA-PEG-NPC to produce polymer PDA-PEG-aGFP, which was fabricated into nanogel NG-aGFP following the same procedures of NG-BSA fabrication. DLS analysis measured a 125.9 nm hydrodynamic diameter (dispersity: 0.20) of nanogel NG-aGFP, see FIG. 3 at A, and negative charges on the surface, see FIG. 8. TEM imaging detected a spherical morphology of NG-aGFP with a diameter of 103.3 nm in dry state, see FIG. 3 at B. The LC and LE values of anti-GFP in NG-aGFP were determined to be 7.6% and 85%, respectively. As shown in FIG. 3 at C, gel electrophoresis demonstrated that the antibody loaded into nanogel NG-aGFP could be effectively recovered by GSH.

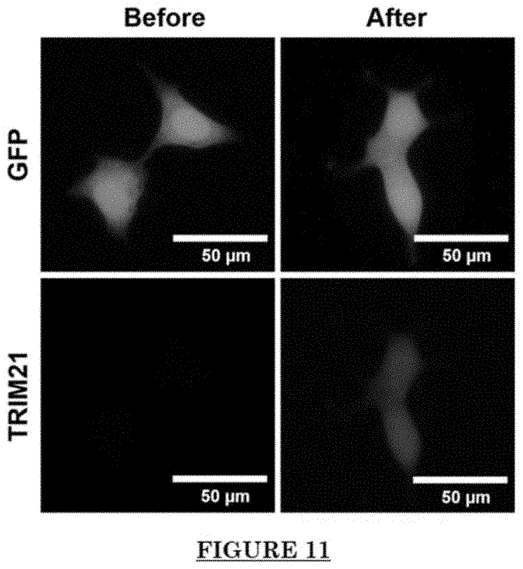

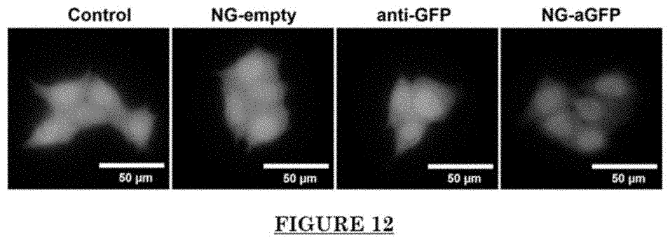

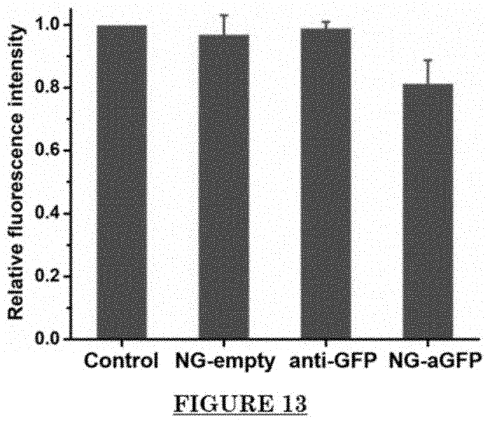

To validate the effectiveness of the Trim-Away technique, we transfected GFP-expressed human breast cancer MCF-7/GFP cells with pmCherry-C1-mTRIM21 plasmid as described in literature, making the cells overexpress TRIM21 protein, since TRIM21 is a pivotal medium for the technique. After transfection, cells emitted cherry fluorescence, see FIG. 11. Then the cells were treated with free anti-GFP antibody, non-loading empty nanogel (NG-empty), and NG-aGFP nanogel at a concentration equivalent to 100 μg/mL anti-GFP. As shown in FIG. 3 at D, the GFP fluorescence significantly decreased in cells incubated with NG-aGFP after 6 h incubation, while no visible change was observed in the fluorescence of cells in other groups, which, along with the quantitative analysis of the fluorescence intensity of cells, see FIG. 3 at E, indicates that the GFP protein of cells was effectively degraded by the intracellular delivery of anti-GFP via NG-aGFP nanogel. We further found that higher concentrations of NG-aGFP yielded better protein degradation efficiency, see FIG. 3 at F, suggesting that the protein degradation is in a dose-dependent manner. As reported, the Trim-Away method relies on the overexpression of protein TRIM21. To verify that, same experiments were conducted in MCF-7/GFP cells without TRIM21-transfection, and obviously, the protein degradation efficiency was considerably limited in comparison to that in TRIM21-transfected cells because of the low endogenous TRIM21 level, see FIGS. 12 and 13, which confirms the necessity of the overexpression of TRIM21 in the protein degradation process.

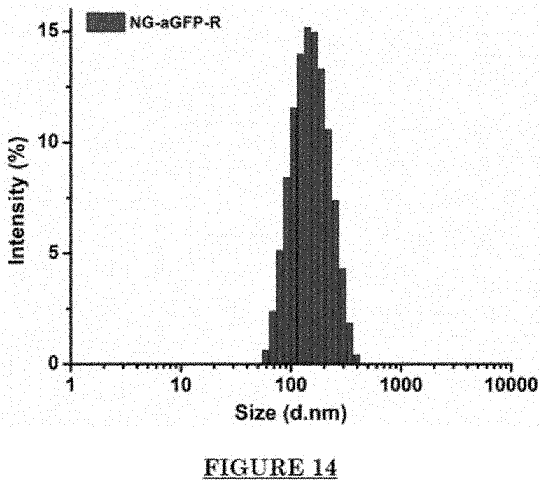

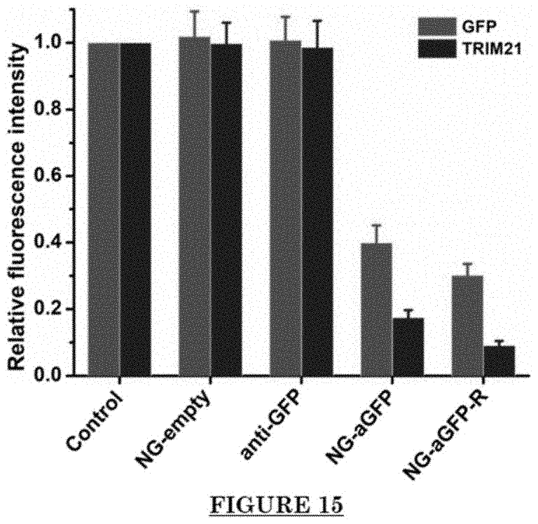

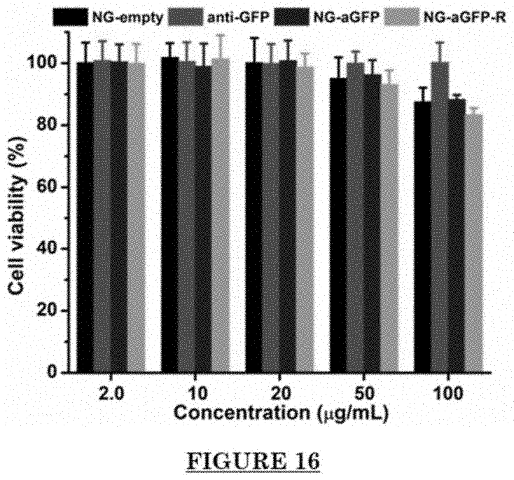

To enhance the cellular uptake of the nanogel, NG-aGFP was decorated with thiol-containing RGD peptide, which can bind to the αᵥβ₃ integrin—a protein receptor overexpressed on a wide spectrum of tumor cells, to yield RGD-modified nanogel NG-aGFP-R. DLS analysis measured a hydrodynamic size of 130.4 nm (dispersity: 0.19) for NG-aGFP-R, see FIG. 14, and zeta potential of the nanogel became less negative after RGD modification, see FIG. 8. As shown in FIG. 3 at G, the weaker GFP fluorescence of cells incubated with NG-aGFP-R, compared to that of cells with NG-aGFP, demonstrated that the nanogel is more efficient in degrading target protein after RGD modification. The quantified analysis of GFP fluorescence intensity further verified the enhanced protein degradation efficiency of NG-aGFP-R nanogel, see FIG. 3 at H. Moreover, the simultaneously decreased fluorescence of both GFP and TRIM21, see FIG. 3 at G, and the corresponding quantitative data, see FIG. 15, indicated the consumption of TRIM21 in the process of protein degradation, which further evidenced the pivotal role of TRIM21 in the Trim-Away technique. Importantly, cell viability test of free anti-GFP and nanogels in TRIM21-transfected MCF-7/GFP cells showed that NG-empty and NG-aGFP nanogels exhibited equal cytotoxicity at same conditions, see FIG. 16, which demonstrated that the nanogel-upgraded Trim-Away induces no additional damage to cells while degrading the target protein.

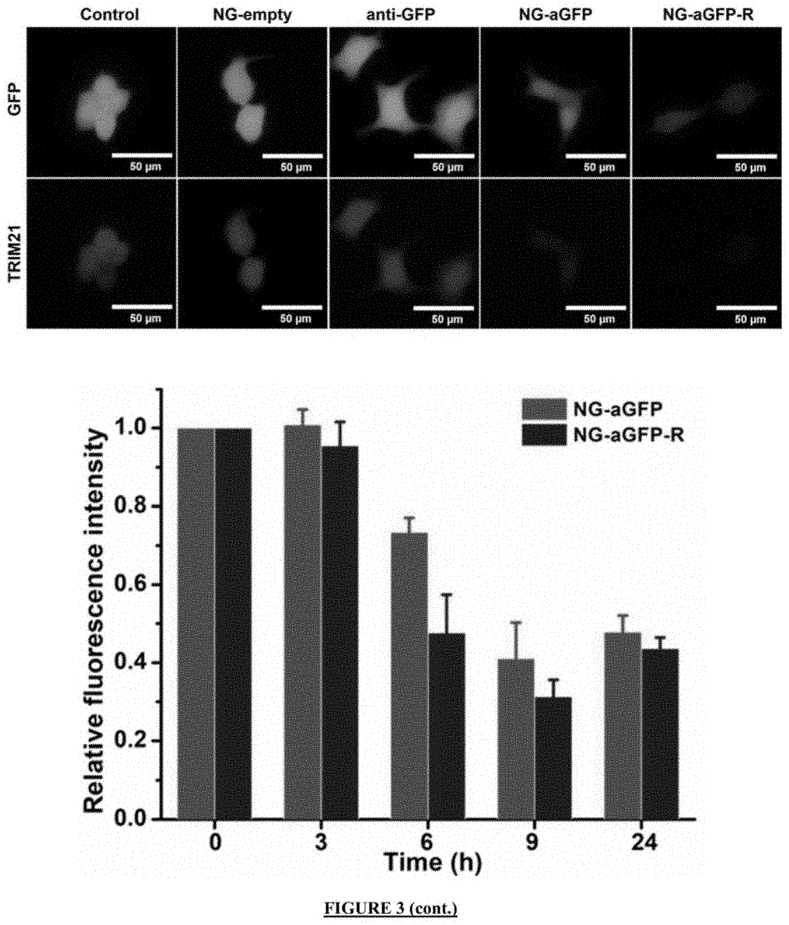

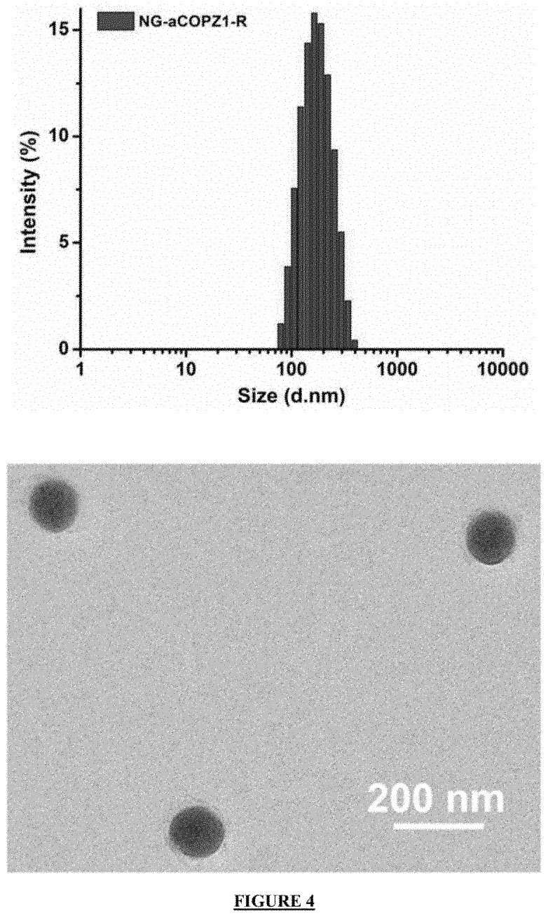

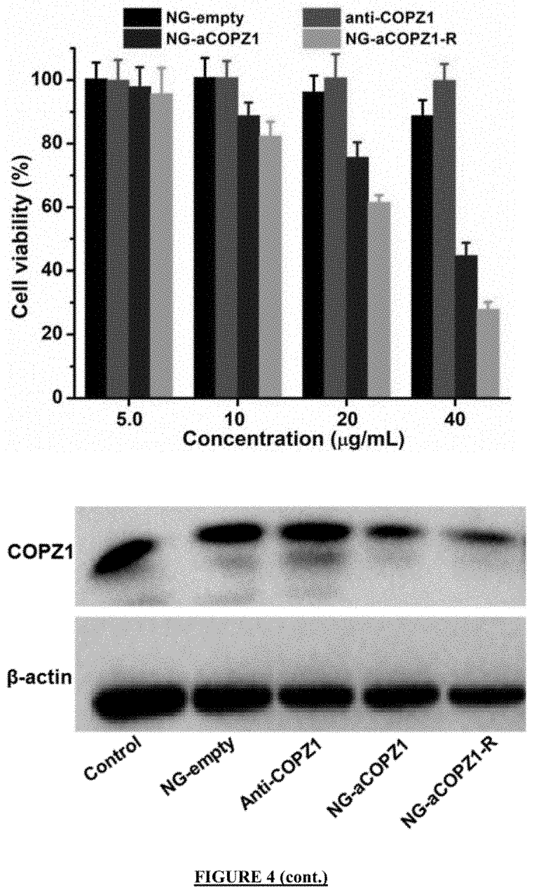

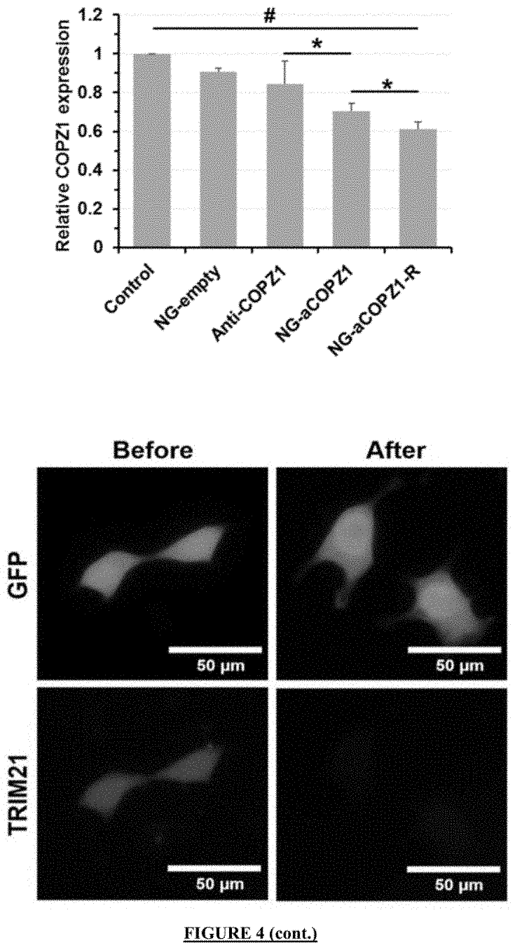

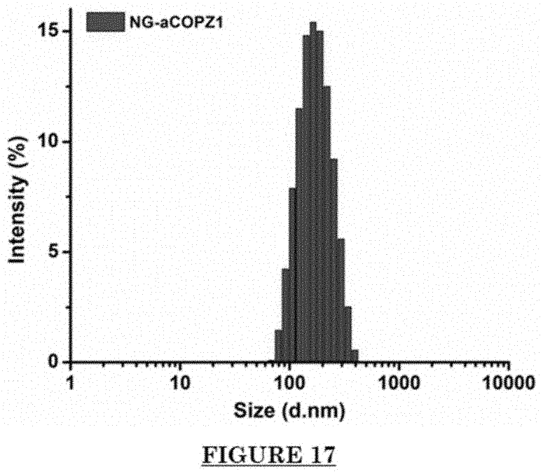

After confirming the effectiveness of the intracellular antibody delivery induced Trim-Away effect, we employed it to degrade a critical protein for cancer cells, the coatomer protein complex 1 (COPZ1) protein, which however is non-essential for normal cells. COPZ1 and COPZ2 are the two isoforms of coatomer protein complex 1 (COPI), which plays vital roles in cells. Normal cells generally express both COPZ1 and COPZ2 proteins, whereas in cancer cells COPZ2 is silenced, and as a result the cells depend solely on COPZ1. Therefore, the degradation of COPZ1 would kill cancer cells, while normal cells survive. The antibody of COPZ1, anti-COPZ1, was loaded into nanogel NG-aCOPZ1 through the same procedures used for NG-BSA and NG-aGFP. DLS analysis showed that NG-aCOPZ1 had a hydrodynamic size of 140.8 nm with a dispersity of 0.21, see FIG. 17, and negative zeta potential, see FIG. 8. The LC and LE values of anti-COPZ1 in NG-aCOPZ1 nanogel were determined to be 3.1% and 81%, respectively. Owing to the significantly enhanced protein degradation efficiency that the RGD ligand brought to NG-aGFP-R nanogel, NG-aCOPZ1 was decorated with RGD as well to produce nanogel NG-aCOPZ1-R. A hydrodynamic diameter of 151.3 nm (dispersity: 0.22) of NG-aCOPZ1-R, see FIG. 4 at A, and a spherical shape with a dry-state size of 121.0 nm was observed by TEM imaging, see FIG. 4 at B. The zeta potential of NG-aCOPZ1-R was less negative in comparison with NG-aCOPZ1, see FIG. 8.

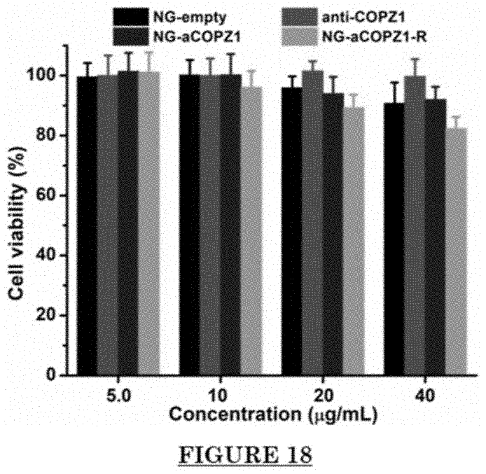

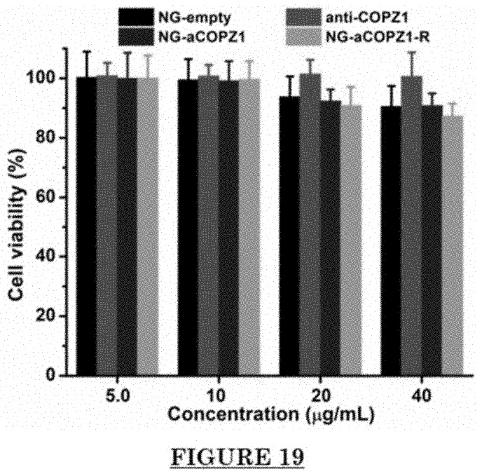

The COPZ1 protein degradation was conducted in MCF-7 cells via evaluating the viability of cells after treatment with anti-COPZ1 loaded nanogels. In MCF-7 cells without TRIM21 transfection, all the nanogels NG-empty, NG-aCOPZ1, and NG-aCOPZ1-R uniformly brought slight inhibitory effect to cell growth when the concentration was high, see FIG. 18, which resulted from the very mild cytotoxicity of the nanogels themselves. However, in TRIM21-transfected MCF-7 cells, as shown in, see FIG. 4 at C, both NG-aCOPZ1 and NG-aCOPZ1-R nanogels noticeably inhibited cell growth in a dose-dependent manner, whereas NG-empty exhibited the same performances as it did in non-transfected MCF-7 cells and free anti-COPZ1 induced no effect towards both TRIM21-transfected and non-transfected cells, which indicated that the anti-COPZ1 loaded nanogels effectively delivered the antibody into cells and successfully degraded the target protein COPZ1 through the Trim-Away path. At a concentration equivalent to 40 μg/mL anti-COPZ1, the viability of cells was decreased to 44.7% and 27.9% by NG-aCOPZ1 and NG-aCOPZ1-R, respectively. The higher Trim-Away efficiency of NG-aCOPZ1-R nanogel should be ascribed to the targeting functionality of RGD ligand. Meanwhile, western blotting analysis showed that the amount of endogenous COPZ1 was considerably diminished in cells incubated with both nanogels, see FIG. 4 at D, as additionally evidenced by the quantified data, see FIG. 4 at E, which further confirmed that the Trim-Away of COPZ1 protein resulted in the suppression of cell viability. Moreover, fluorescence images of TRIM21-transfected MCF-7/GFP cells before and after incubation with NG-aCOPZ1-R nanogel showed that TRIM21 was largely consumed while GFP protein was barely affected in the process of the Trim-Away of COPZ1 protein, see FIG. 4 at F, demonstrating the high specificity of the upgraded Trim-Away technique in degrading the target protein. In addition, the application of Trim-Away with anti-COPZ1 nanogels in TRIM21-transfected NIH-3T3 cells caused negligible damage to cells, see FIG. 19, suggesting that this Trim-Away approach is safe for normal cells. All the results collectively confirm that the upgraded Trim-Away technique is efficient for degrading endogenous proteins and is potential to be exploited as a protein therapy modality for cancer treatment.

Further, the protein-loaded nanogel may include antibodies and/or nanobodies. The antibodies may include anti COPZ1 antibody, anti PTBP1 antibody, anti PD-L1 antibody, anti PD-1 antibody, anti-Her2 antibody, anti EGFR antibody, anti survivin antibody, anti PTP1B antibody, anti VEGF antibody, anti PKN3 antibody. The nanobodies may include anti COPZ1 nanobody, anti PTBP1 nanobody, anti PD-L1 nanobody, anti PD-1 nanobody, anti-Her2 nanobody, anti EGFR nanobody, anti survivin nanobody, anti PTP1B nanobody, anti VEGF nanobody, anti PKN3 nanobody.

### CONCLUSION

In summary, protein/antibody covalently loaded redox-responsive polymer nanogels have been fabricated for intracellular delivery and traceless release of protein/antibody, and based on which, a new intracellular antibody delivery method has been developed for degrading a specific endogenous protein. The proteins/antibodies are conjugated to the nanogels via a redox-sensitive self-immolative linker, which can be cleaved by intracellular GSH and release the proteins/antibodies in a traceless form. After being delivered into cells, the unloaded antibody binds to its target protein and Trim-21, and subsequently degrades through TRIM21-mediated ubiquitination in the proteasome. The nano-Trim-Away technique has been proven highly efficient in degrading endogenous proteins with more convenience and wide-application potency compared with its original version. Notably, this new technique has been successfully employed to degrade a vital protein COPZ1 for cancer cells and kill the cells as a result, without causing damage to normal cells. In a word, the nanogel-Trim-Away technique is promising to provide a reliable and convenient tool for endogenous protein study and to arouse the emergence of new protein/antibody-based therapeutic modalities for cancer and other diseases, including but not limited to Alzheimer's diseases, Parkinson's disease, multiple sclerosis, neonatal hypoxic-ischemic, stroke, Amyotrophic lateral sclerosis, Huntington's disease, spinal cord injury, brain injury, retina injury, post-traumatic stress disorder, and frontotemporal dementia, and traumatic brain injury

Sequence Listings

| | |
|---|---|
| <110> | University of South Carolina |
| <120> | A Method For The Degradation Of Endogenous Protein |
| <130> | 2033101.0000288 |
| <140> | Unknown |
| <141> | 2021-04-28 |
| <150> | U.S. Provisional Application No. 63/045,275 |
| <151> | 06/29/2020 |
| <160> | 12 |
| <170> | PatentIn |
| <210> | 1 |
| <211> | 239 |
| <212> | PRT |
| <213> | Homo sapiens |
| <221> | CDS |
| <222> | 1...239 EGFP |
| <400> | 1 |

```
Met Val Ser Lys Gly Glu Glu Leu Phe Thr Gly Val Pro Ile Leu
1 5 10 15
Val Glu Leu Asp Gly Asp Val Asn Gly His Lys Phe Ser Val Ser Gly
                        20 25 30
Glu Gly Glu Gly Asp Ala Thr Tyr Gly Lys Leu Thr Leu Lys Phe Ile
                35 40 45
Cys Thr Thr Gly Lys Leu Pro Val Pro Trp Pro Thr Leu Val Thr Thr
        50 55 60
Leu Thr Tyr Gly Val Gln Cys Phe Ser Arg Tyr Pro Asp His Met Lys
65 70 75 80
Gln His Asp Phe Phe Lys Ser Ala Met Pro Glu Gly Tyr Val Gln Glu
                                85 90 95
Arg Thr Ile Phe Phe Lys Asp Asp Gly Asn Tyr Lys Thr Arg Ala Glu
                        100 105 110
Val Lys Phe Glu Gly Asp Thr Leu Val Asn Arg Ile Glu Leu Lys Gly
                115 120 125
Ile Asp Phe Lys Glu Asp Gly Asn Ile Leu Gly His Lys Leu Glu Tyr
        130 135 140
Asn Tyr Asn Ser His Asn Val Tyr Ile Met Ala Asp Lys Gln Lys Asn
145 150 155 160
Gly Ile Lys Val Asn Phe Lys Ile Arg His Asn Ile Glu Asp Gly Ser
                                165 170 175
Val Gln Leu Ala Asp His Tyr Gln Gln Asn Thr Pro Ile Gly Asp Gly
                        180 185 190
Pro Val Leu Leu Pro Asp Asn His Tyr Leu Ser Thr Gln Ser Ala Leu
                195 200 205
Ser Lys Asp Pro Asn Gln Lys Arg Asp His Met Val Leu Leu Gln Phe
        210 215 220
Val Thr Ala Ala Gly Ile Thr Leu Gly Met Asp Glu Leu Tyr Lys
225 230 235
```

| | |
|---|---|
| <210> | 2 |
| <211> | 475 |
| <212> | PRT |
| <213> | Homo sapiens |
| <222> | 1...475 TRIM21 |
| <400> | 2 |

```
Met Ala Ser Ala Ala Arg Leu Thr Met Met Trp Glu Glu Val Thr Cys
1 5 10 15
Pro Ile Cys Leu Asp Pro Phe Val Glu Pro Val Ser Ile Glu Cys Gly
                        20 25 30
His Ser Phe Cys Gln Glu Cys Ile Ser Gln Val Gly Lys Gly Gly Gly
                35 40 45
Ser Val Cys Pro Val Cys Arg Gln Arg Phe Leu Leu Lys Asn Leu Arg
        50 55 60
Pro Asn Arg Gln Leu Ala Asn Met Val Asn Asn Leu Lys Glu Ile Ser
65 70 75 80
Gln Glu Ala Arg Glu Gly Thr Gln Gly Glu Arg Cys Ala Val His Gly
                                85 90 95
Glu Arg Leu His Leu Phe Cys Glu Lys Asp Gly Lys Ala Leu Cys Trp
                        100 105 110
Val Cys Ala Gln Ser Arg Lys His Arg Asp His Ala Met Val Pro Leu
                115 120 125
Glu Glu Ala Ala Gln Glu Tyr Gln Glu Lys Leu Gln Val Ala Leu Gly
        130 135 140
Glu Leu Arg Arg Lys Gln Glu Leu Ala Glu Lys Leu Glu Val Glu Ile
145 150 155 160
Ala Ile Lys Arg Ala Asp Trp Lys Lys Thr Val Glu Thr Gln Lys Ser
                                165 170 175
Arg Ile His Ala Glu Phe Val Gln Gln Lys Asn Phe Leu Val Glu Glu
                        180 185 190
Glu Gln Arg Gln Leu Gln Glu Leu Glu Lys Asp Glu Arg Glu Gln Leu
                195 200 205
Arg Ile Leu Gly Glu Lys Glu Ala Lys Leu Ala Gln Gln Ser Gln Ala
        210 215 220
Leu Gln Glu Leu Ile Ser Glu Leu Asp Arg Arg Cys His Ser Ser Ala
225 230 235 240
Leu Glu Leu Leu Gln Glu Val Ile Ile Val Leu Glu Arg Ser Glu Ser
                                245 250 255
Trp Asn Leu Lys Asp Leu Asp Ile Thr Ser Pro Glu Leu Arg Ser Val
                        260 265 270
Cys His Val Pro Gly Leu Lys Lys Met Leu Arg Thr Cys Ala Val His
                275 280 285
Ile Thr Leu Asp Pro Asp Thr Ala Asn Pro Trp Leu Ile Leu Ser Glu
        290 295 300
Asp Arg Arg Gln Val Arg Leu Gly Asp Thr Gln Gln Ser Ile Pro Gly
305 310 315 320
Asn Glu Glu Arg Phe Asp Ser Tyr Pro Met Val Leu Gly Ala Gln His
                                325 330 335
Phe His Ser Gly Lys His Tyr Trp Glu Val Asp Val Thr Gly Lys Glu
                        340 345 350
Ala Trp Asp Leu Gly Val Cys Arg Asp Ser Val Arg Arg Lys Gly His
                355 360 365
Phe Leu Leu Ser Ser Lys Ser Gly Phe Trp Thr Ile Trp Leu Trp Asn
        370 375 380
Lys Gln Lys Tyr Glu Ala Gly Thr Tyr Pro Gln Thr Pro Leu His Leu
385 390 395 400
Gln Val Pro Pro Cys Gln Val Gly Ile Phe Leu Asp Tyr Glu Ala Gly
                                405 410 415
Met Val Ser Phe Tyr Asn Ile Thr Asp His Gly Ser Leu Ile Tyr Ser
                        420 425 430
Phe Ser Glu Cys Ala Phe Thr Gly Pro Leu Arg Pro Phe Phe Ser Pro
                435 440 445
Gly Phe Asn Asp Gly Gly Lys Asn Thr Ala Pro Leu Thr Leu Cys Pro
        450 455 460
Leu Asn Ile Gly Ser Gln Gly Ser Thr Asp Tyr
465 470 475
```

| | |
|---|---|
| <210> | 3 |
| <211> | 475 |
| <212> | PRT |
| <213> | Homo sapiens |
| <222> | 1...177 COPZ1 |
| <400> | 3 |

```
Met Glu Ala Leu Ile Leu Glu Pro Ser Leu Tyr Thr Val Lys Ala Ile
1 5 10 15
Leu Ile Leu Asp Asn Asp Gly Asp Arg Leu Phe Ala Lys Tyr Tyr Asp
                        20 25 30
Asp Thr Tyr Pro Ser Val Lys Glu Gln Lys Ada Phe Glu Lys Asn Ile
                35 40 45
Phe Asn Lys Thr His Arg Thr Asp Ser Glu Ile Ala Leu Leu Glu Gly
        50 55 60
Leu Thr Val Val Tyr Lys Ser Ser Ile Asp Leu Tyr Phe Tyr Val Ile
65 70 75 80
Gly Ser Ser Tyr Glu Asn Glu Leu Met Leu Met Ala Val Leu Asn Cys
                                85 90 95
Leu Phe Asp Ser Leu Ser Gln Met Leu Arg Lys Asn Val Glu Lys Arg
                        100 105 110
Ala Leu Leu Glu Asn Met Glu Gly Leu Phe Leu Ala Val Asp Glu Ile
                115 120 125
Val Asp Gly Gly Val Ile Leu Glu Ser Asp Pro Gln Gln Val Val His
        130 135 140
Arg Val Ala Leu Arg Gly Glu Asp Val Pro Leu Thr Glu Gln Thr Val
145 150 155 160
Ser Gln Val Leu Gln Ser Ala Lys Glu Gln Ile Lys Trp Ser Leu Leu
                                165 170 175
Arg
```

| | |
|---|---|
| <210> | 4 |
| <211> | 531 |
| <212> | PRT |
| <213> | Homo sapiens |
| <400> | 4 |

```
Met Asp Gly Ile Val Pro Asp Ile Ala Val Gly Thr Lys Arg Gly Ser
1 5 10 15
Asp Glu Leu Phe Ser Thr Cys Val Thr Asn Gly Pro Phe Ile Met Ser
                        20 25 30
Ser Asn Ser Ala Ser Ala Ala Asn Gly Asn Asp Ser Lys Lys Phe Lys
                35 40 45
Gly Asp Ser Arg Ser Ala Gly Val Pro Ser Arg Val Ile His Ile Arg
        50 55 60
Lys Leu Pro Ile Asp Val Thr Glu Gly Glu Val Ile Ser Leu Gly Leu
65 70 75 80
Pro Phe Gly Lys Val Thr Asn Leu Leu Met Leu Lys Gly Lys Asn Gln
                                85 90 95
Ala Phe Ile Glu Met Asn Thr Glu Glu Ala Ala Asn Thr Met Val Asn
                        100 105 110
Tyr Tyr Thr Ser Val Thr Pro Val Leu Arg Gly Gln Pro Ile Tyr Ile
                115 120 125
Gln Phe Ser Asn His Lys Glu Leu Lys Thr Asp Ser Ser Pro Asn Gln
        130 135 140
Ala Arg Ala Gln Ala Ala Leu Gln Ala Val Asn Ser Val Gln Ser Gly
145 150 155 160
Asn Leu Ala Leu Ala Ala Ser Ala Ala Ala Val Asp Ala Gly Met Ala
                                165 170 175
Met Ala Gly Gln Ser Pro Val Leu Arg Ile Ile Val Glu Asn Leu Phe
                        180 185 190
Tyr Pro Val Thr Leu Asp Val Leu His Gln Ile Phe Ser Lys Phe Gly
                195 200 205
Thr Val Leu Lys Ile Ile Thr Phe Thr Lys Asn Asn Gln Phe Gln Ala
        210 215 220
Leu Leu Gln Tyr Ala Asp Pro Val Ser Ala Gln His Ala Lys Leu Ser
225 230 235 240
Leu Asp Gly Gln Asn Ile Tyr Asn Ala Cys Cys Thr Leu Arg Ile Asp
                                245 250 255
Phe Ser Lys Leu Thr Ser Leu Asn Val Lys Tyr Asn Asn Asp Lys Ser
                        260 265 270
Arg Asp Tyr Thr Arg Pro Asp Leu Pro Ser Gly Asp Ser Gln Pro Ser
                275 280 285
Leu Asp Gln Thr Met Ala Ala Ala Phe Gly Leu Ser Val Pro Asn Val
        290 295 300
His Gly Ala Leu Ala Pro Leu Ala Ile Pro Ser Ala Ala Ala Ala Ala
305 310 315 320
Ala Ala Ala Gly Arg Ile Ala Ile Pro Gly Leu Ala Gly Ala Gly Asn
                                325 330 335
Ser Val Leu Leu Val Ser Asn Leu Asn Pro Glu Arg Val Thr Pro Gln
                        340 345 350
Ser Leu Phe Ile Leu Phe Gly Val Tyr Gly Asp Val Gln Arg Val Lys
                355 360 365
Ile Leu Phe Asn Lys Lys Glu Asn Ala Leu Val Gln Met Ala Asp Gly
        370 375 380
Asn Gln Ala Gln Leu Ala Met Ser His Leu Asn Gly His Lys Leu His
385 390 395 400
Gly Lys Pro Ile Arg Ile Thr Leu Ser Lys His Gln Asn Val Gln Leu
                                405 410 415
Pro Arg Glu Gly Gln Glu Asp Gln Gly Leu Thr Lys Asp Tyr Gly Asn
                        420 425 430
Ser Pro Leu His Arg Phe Lys Lys Pro Gly Ser Lys Asn Phe Gln Asn
                435 440 445
Ile Phe Pro Pro Ser Ala Thr Leu His Leu Ser Asn Ile Pro Pro Ser
        450 455 460
Val Ser Glu Glu Asp Leu Lys Val Leu Phe Ser Ser Asn Gly Gly Val
465 470 475 480
Val Lys Gly Phe Lys Phe Phe Gln Lys Asp Arg Lys Met Ala Leu Ile
                                485 490 495
Gln Met Gly Ser Val Glu Glu Ala Val Gln Ala Leu Ile Asp Leu His
                        500 505 510
Asn His Asp Leu Gly Glu Asn His His Leu Arg Val Ser Phe Ser Lys
                515 520 525
Ser Thr Ile
        530
```

| | |
|---|---|
| <210> | 5 |
| <211> | 290 |
| <212> | PRT |
| <213> | Homo sapiens |
| <400> | 5 |

```
Met Arg Ile Phe Ala Val Phe Ile Phe Met Thr Tyr Trp His Leu Leu
1 5 10 15
Asn Ala Phe Thr Val Thr Val Pro Lys Asp Leu Tyr Val Val Glu Tyr
                        20 25 30
Gly Ser Asn Met Thr Ile Glu Cys Lys Phe Pro Val Glu Lys Gln Leu
                35 40 45
Asp Leu Ala Ala Leu Ile Val Tyr Trp Glu Met Glu Asp Lys Asn Ile
        50 55 60
Ile Gln Phe Val His Gly Glu Glu Asp Leu Lys Val Gln His Ser Ser
65 70 75 80
Tyr Arg Gln Arg Ala Arg Leu Leu Lys Asp Gln Leu Ser Leu Gly Asn
                                85 90 95
Ala Ala Leu Gln Ile Thr Asp Val Lys Leu Gln Asp Ala Gly Val Tyr
                        100 105 110
Arg Cys Met Ile Ser Tyr Gly Gly Ala Asp Tyr Lys Arg Ile Thr Val
                115 120 125
Lys Val Asn Ala Pro Tyr Asn Lys Ile Asn Gln Arg Ile Leu Val Val
        130 135 140
Asp Pro Val Thr Ser Glu His Glu Leu Thr Cys Gln Ala Glu Gly Tyr
145 150 155 160
Pro Lys Ala Glu Val Ile Trp Thr Ser Ser Asp His Gln Val Leu Ser
                                165 170 175
Gly Lys Thr Thr Thr Thr Asn Ser Lys Arg Glu Glu Lys Leu Phe Asn
                        180 185 190
Val Thr Ser Thr Leu Arg Ile Asn Thr Thr Thr Asn Glu Ile Phe Tyr
                195 200 205
Cys Thr Phe Arg Arg Leu Asp Pro Glu Glu Asn His Thr Ala Glu Leu
        210 215 220
Val Ile Pro Glu Leu Pro Leu Ala His Pro Pro Asn Glu Arg Thr His
225 230 235 240
Leu Val Ile Leu Gly Ala Ile Leu Leu Cys Leu Gly Val Ala Leu Thr
                                245 250 255
Phe Ile Phe Arg Leu Arg Lys Gly Arg Met Met Asp Val Lys Lys Cys
                        260 265 270
Gly Ile Gln Asp Thr Asn Ser Lys Lys Gln Ser Asp Thr His Leu Glu
                275 280 285
Glu Thr
        290
```

| | |
|---|---|
| <210> | 6 |
| <211> | 288 |
| <212> | PRT |
| <213> | Homo sapiens |
| <400> | 6 |

```
Met Gln Ile Pro Gln Ala Pro Trp Pro Val Val Trp Ala Val Leu Gln
1 5 10 15
Leu Gly Trp Arg Pro Gly Trp Phe Leu Asp Ser Pro Asp Arg Pro Trp
                        20 25 30
Asn Pro Pro Thr Phe Ser Pro Ala Leu Leu Val Val Thr Glu Gly Asp
                35 40 45
Asn Ala Thr Phe Thr Cys Ser Phe Ser Asn Thr Ser Glu Ser Phe Val
        50 55 60
Leu Asn Trp Tyr Arg Met Ser Pro Ser Asn Gln Thr Asp Lys Leu Ala
65 70 75 80
Ala Phe Pro Glu Asp Arg Ser Gln Pro Gly Gln Asp Cys Arg Phe Arg
                                85 90 95
Val Thr Gln Leu Pro Asn Gly Arg Asp Phe His Met Ser Val Val Arg
                        100 105 110
Ala Arg Arg Asn Asp Ser Gly Thr Tyr Leu Cys Gly Ala Ile Ser Leu
                115 120 125
Ala Pro Lys Ala Gln Ile Lys Glu Ser Leu Arg Ala Glu Leu Arg Val
        130 135 140
Thr Glu Arg Arg Ala Glu Val Pro Thr Ala His Pro Ser Pro Ser Pro
145 150 155 160
Arg Pro Ala Gly Gln Phe Gln Thr Leu Val Val Gly Val Val Gly Gly
                                165 170 175
Leu Leu Gly Ser Leu Val Leu Leu Val Trp Val Leu Ala Val Ile Cys
                        180 185 190
Ser Arg Ala Ala Arg Gly Thr Ile Gly Ala Arg Arg Thr Gly Gln Pro
                195 200 205
Leu Lys Glu Asp Pro Ser Ala Val Pro Val Phe Ser Val Asp Tyr Gly
        210 215 220
Glu Leu Asp Phe Gln Trp Arg Glu Lys Thr Pro Glu Pro Pro Val Pro
225 230 235 240
Cys Val Pro Glu Gln Thr Glu Tyr Ala Thr Ile Val Phe Pro Ser Gly
                                245 250 255
Met Gly Thr Ser Ser Pro Ala Arg Arg Gly Ser Ala Asp Gly Pro Arg
                        260 265 270
Ser Ala Gln Pro Leu Arg Pro Glu Asp Gly His Cys Ser Trp Pro Leu
                275 280 285
```

| | |
|---|---|
| <210> | 7 |
| <211> | 1255 |
| <212> | PRT |
| <213> | Homo sapiens |
| <400> | 7 |

```
Met Glu Leu Ala Ala Leu Cys Arg Trp Gly Leu Leu Leu Ala Leu Leu
1 5 10 15
Pro Pro Gly Ala Ala Ser Thr Gln Val Cys Thr Gly Thr Asp Met Lys
                        20 25 30
Leu Arg Leu Pro Ala Ser Pro Glu Thr His Leu Asp Met Leu Arg His
                35 40 45
Leu Tyr Gln Gly Cys Gln Val Val Gln Gly Asn Leu Glu Leu Thr Tyr
        50 55 60
Leu Pro Thr Asn Ala Ser Leu Ser Phe Leu Gln Asp Ile Gln Glu Val
65 70 75 80
Gln Gly Tyr Val Leu Ile Ala His Asn Gln Val Arg Gln Val Pro Leu
                                85 90 95
Gln Arg Leu Arg Ile Val Arg Gly Thr Gln Leu Phe Glu Asp Asn Tyr
                        100 105 110
Ala Leu Ala Val Leu Asp Asn Gly Asp Pro Leu Asn Asn Thr Thr Pro
                115 120 125
Val Thr Gly Ala Ser Pro Gly Gly Leu Arg Glu Leu Gln Leu Arg Ser
        130 135 140
Leu Thr Glu Ile Leu Lys Gly Gly Val Leu Ile Gln Arg Asn Pro Gln
145 150 155 160
Leu Cys Tyr Gln Asp Thr Ile Leu Trp Lys Asp Ile Phe His Lys Asn
                                165 170 175
Asn Gln Leu Ala Leu Thr Leu Ile Asp Thr Asn Arg Ser Arg Ala Cys
                        180 185 190
His Pro Cys Ser Pro Met Cys Lys Gly Ser Arg Cys Trp Gly Glu Ser
                195 200 205
Ser Glu Asp Cys Gln Ser Leu Thr Arg Thr Val Cys Ala Gly Gly Cys
        210 215 220
Ala Arg Cys Lys Gly Pro Leu Pro Thr Asp Cys Cys His Glu Gln Cys
225 230 235 240
Ada Ala Gly Cys Thr Gly Pro Lys His Ser Asp Cys Leu Ala Cys Leu
                                245 250 255
His Phe Asn His Ser Gly Ile Cys Glu Leu His Cys Pro Ala Leu Val
                        260 265 270
Thr Tyr Asn Thr Asp Thr Phe Glu Ser Met Pro Asn Pro Glu Gly Arg
                275 280 285
Tyr Thr Phe Gly Ala Ser Cys Val Thr Ala Cys Pro Tyr Asn Tyr Leu
        290 295 300
Ser Thr Asp Val Gly Ser Cys Thr Leu Val Cys Pro Leu His Asn Gln
305 310 315 320
Glu Val Thr Ala Glu Asp Gly Thr Gln Arg Cys Glu Lys Cys Ser Lys
                                325 330 335
Pro Cys Ala Arg Val Cys Tyr Gly Leu Gly Met Glu His Leu Arg Glu
                        340 345 350
Val Arg Ala Val Thr Ser Ala Asn Ile Gln Glu Phe Ala Gly Cys Lys
                355 360 365
Lys Ile Phe Gly Ser Leu Ala Phe Leu Pro Glu Ser Phe Asp Gly Asp
        370 375 380
Pro Ala Ser Asn Thr Ala Pro Leu Gln Pro Glu Gln Leu Gln Val Phe
385 390 395 400
Glu Thr Leu Glu Glu Ile Thr Gly Tyr Leu Tyr Ile Ser Ala Trp Pro
                                405 410 415
Asp Ser Leu Pro Asp Leu Ser Val Phe Gln Asn Leu Gln Val Ile Arg
                        420 425 430
Gly Arg Ile Leu His Asn Gly Ala Tyr Ser Leu Thr Leu Gln Gly Leu
                435 440 445
Gly Ile Ser Trp Leu Gly Leu Arg Ser Leu Arg Glu Leu Gly Ser Gly
        450 455 460
Leu Ala Leu Ile His His Asn Thr His Leu Cys Phe Val His Thr Val
465 470 475 480
Pro Trp Asp Gln Leu Phe Arg Asn Pro His Gln Ala Leu Leu His Thr
                                485 490 495
Ala Asn Arg Pro Glu Asp Glu Cys Val Gly Glu Gly Leu Ala Cys His
                        500 505 510
Gln Leu Cys Ala Arg Gly His Cys Trp Gly Pro Gly Pro Thr Gln Cys
                515 520 525
Val Asn Cys Ser Gln Phe Leu Arg Gly Gln Glu Cys Val Glu Glu Cys
        530 535 540
Arg Val Leu Gln Gly Leu Pro Arg Glu Tyr Val Asn Ala Arg His Cys
545 550 555 560
Leu Pro Cys His Pro Glu Cys Gln Pro Gln Asn Gly Ser Val Thr Cys
                                565 570 575
Phe Gly Pro Glu Ala Asp Gln Cys Val Ala Cys Ala His Tyr Lys Asp
                        580 585 590
Pro Pro Phe Cys Val Ala Arg Cys Pro Ser Gly Val Lys Pro Asp Leu
                595 600 605
Ser Tyr Met Pro Ile Trp Lys Phe Pro Asp Glu Glu Gly Ala Cys Gln
        610 615 620
Pro Cys Pro Ile Asn Cys Thr His Ser Cys Val Asp Leu Asp Asp Lys
625 630 635 640
Gly Cys Pro Ala Glu Gln Arg Ala Ser Pro Leu Thr Ser Ile Ile Ser
                                645 650 655
Ala Val Val Gly Ile Leu Leu Val Val Val Leu Gly Val Val Phe Gly
                        660 665 670
Ile Leu Ile Lys Arg Arg Gln Gln Lys Ile Arg Lys Tyr Thr Met Arg
                675 680 685
Arg Leu Leu Gln Glu Thr Glu Leu Val Glu Pro Leu Thr Pro Ser Gly
        690 695 700
Ala Met Pro Asn Gln Ala Gln Met Arg Ile Leu Lys Glu Thr Glu Leu
705 710 715 720
Arg Lys Val Lys Val Leu Gly Ser Gly Ala Phe Gly Thr Val Tyr Lys
                                725 730 735
Gly Ile Trp Ile Pro Asp Gly Glu Asn Val Lys Ile Pro Val Ala Ile
                        740 745 750
Lys Val Leu Arg Glu Asn Thr Ser Pro Lys Ala Asn Lys Glu Ile Leu
                755 760 765
Asp Glu Ala Tyr Val Met Ala Gly Val Gly Ser Pro Tyr Val Ser Arg
        770 775 780
Leu Leu Gly Ile Cys Leu Thr Ser Thr Val Gln Leu Val Thr Gln Leu
785 790 795 800
Met Pro Tyr Gly Cys Leu Leu Asp His Val Arg Glu Asn Arg Gly Arg
                                805 810 815
Leu Gly Ser Gln Asp Leu Leu Asn Trp Cys Met Gln Ile Ala Lys Gly
                        820 825 830
Met Ser Tyr Leu Glu Asp Val Arg Leu Val His Arg Asp Leu Ala Ala
                835 840 845
Arg Asn Val Leu Val Lys Ser Pro Asn His Val Lys Ile Thr Asp Phe
        850 855 860
Gly Leu Ala Arg Leu Leu Asp Ile Asp Glu Thr Glu Tyr His Ala Asp
865 870 875 880
Gly Gly Lys Val Pro Ile Lys Trp Met Ala Leu Glu Ser Ile Leu Arg
                                885 890 895
Arg Arg Phe Thr His Gln Ser Asp Val Trp Ser Tyr Gly Val Thr Val
                        900 905 910
Trp Glu Leu Met Thr Phe Gly Ala Lys Pro Tyr Asp Gly Ile Pro Ala
                915 920 925
Arg Glu Ile Pro Asp Leu Leu Glu Lys Gly Glu Arg Leu Pro Gln Pro
        930 935 940
Pro Ile Cys Thr Ile Asp Val Tyr Met Ile Met Val Lys Cys Trp Met
945 950 955 960
Ile Asp Ser Glu Cys Arg Pro Arg Phe Arg Glu Leu Val Ser Glu Phe
                                965 970 975
Ser Arg Met Ala Arg Asp Pro Gln Arg Phe Val Val Ile Gln Asn Glu
                        980 985 990
Asp Leu Gly Pro Ala Ser Pro Leu Asp Ser Thr Phe Tyr Arg Ser Leu
                995 1000 1005
Leu Glu Asp Asp Asp Met Gly Asp Leu Val Asp Ala Glu Glu Tyr
        1010 1015 1020
Leu Val Pro Gln Gln Gly Phe Phe Cys Pro Asp Pro Ala Pro Gly
        1025 1030 1035
Ala Gly Gly Met Val His His Arg His Arg Ser Ser Ser Thr Arg
        1040 1045 1050
Ser Gly Gly Gly Asp Leu Thr Leu Gly Leu Glu Pro Ser Glu Glu
        1055 1060 1065
Glu Ala Pro Arg Ser Pro Leu Ala Pro Ser Glu Gly Ala Gly Ser
        1070 1075 1080
Asp Val Phe Asp Gly Asp Leu Gly Met Gly Ala Ala Lys Gly Leu
        1085 1090 1095
Gln Ser Leu Pro Thr His Asp Pro Ser Pro Leu Gln Arg Tyr Ser
        1100 1105 1110
Glu Asp Pro Thr Val Pro Leu Pro Ser Glu Thr Asp Gly Tyr Val
        1115 1120 1125
Ala Pro Leu Thr Cys Ser Pro Gln Pro Glu Tyr Val Asn Gln Pro
        1130 1135 1140
Asp Val Arg Pro Gln Pro Pro Ser Pro Arg Glu Gly Pro Leu Pro
        1145 1150 1155
Ala Ala Arg Pro Ala Gly Ala Thr Leu Glu Arg Pro Lys Thr Leu
        1160 1165 1170
Ser Pro Gly Lys Asn Gly Val Val Lys Asp Val Phe Ala Phe Gly
        1175 1180 1185
Gly Ala Val Glu Asn Pro Glu Tyr Leu Thr Pro Gln Gly Gly Ala
        1190 1195 1200
Ala Pro Gln Pro His Pro Pro Pro Ala Phe Ser Pro Ala Phe Asp
        1205 1210 1215
Asn Leu Tyr Tyr Trp Asp Gln Asp Pro Pro Glu Arg Gly Ala Pro
        1220 1225 1230
Pro Ser Thr Phe Lys Gly Thr Pro Thr Ala Glu Asn Pro Glu Tyr
        1235 1240 1245
Leu Gly Leu Asp Val Pro Val
        1250 1255
```

| | |
|---|---|
| <210> | 8 |
| <211> | 1210 |
| <212> | PRT |
| <213> | Homo sapiens |
| <400> | 8 |

```
Met Arg Pro Ser Gly Thr Ala Gly Ala Ala Leu Leu Ala Leu Leu Ala
1 5 10 15
Ala Leu Cys Pro Ala Ser Arg Ala Leu Glu Glu Lys Lys Val Cys Gln
                        20 25 30
Gly Thr Ser Asn Lys Leu Thr Gln Leu Gly Thr Phe Glu Asp His Phe
                35 40 45
Leu Ser Leu Gln Arg Met Phe Asn Asn Cys Glu Val Val Leu Gly Asn
        50 55 60
Leu Glu Ile Thr Tyr Val Gln Arg Asn Tyr Asp Leu Ser Phe Leu Lys
65 70 75 80
Thr Ile Gln Glu Val Ala Gly Tyr Val Leu Ile Ala Leu Asn Thr Val
                                85 90 95
Glu Arg Ile Pro Leu Glu Asn Leu Gln Ile Ile Arg Gly Asn Met Tyr
                        100 105 110
Tyr Glu Asn Ser Tyr Ala Leu Ala Val Leu Ser Asn Tyr Asp Ala Asn
                115 120 125
Lys Thr Gly Leu Lys Glu Leu Pro Met Arg Asn Leu Gln Glu Ile Leu
        130 135 140
His Gly Ala Val Arg Phe Ser Asn Asn Pro Ala Leu Cys Asn Val Glu
145 150 155 160
Ser Ile Gln Trp Arg Asp Ile Val Ser Ser Asp Phe Leu Ser Asn Met
                                165 170 175
Ser Met Asp Phe Gln Asn His Leu Gly Ser Cys Gln Lys Cys Asp Pro
                        180 185 190
Ser Cys Pro Asn Gly Ser Cys Trp Gly Ala Gly Glu Glu Asn Cys Gln
                195 200 205
Lys Leu Thr Lys Ile Ile Cys Ala Gln Gln Cys Ser Gly Arg Cys Arg
        210 215 220
Gly Lys Ser Pro Ser Asp Cys Cys His Asn Gln Cys Ala Ala Gly Cys
225 230 235 240
Thr Gly Pro Arg Glu Ser Asp Cys Leu Val Cys Arg Lys Phe Arg Asp
                                245 250 255
Glu Ala Thr Cys Lys Asp Thr Cys Pro Pro Leu Met Leu Tyr Asn Pro
                        260 265 270
Thr Thr Tyr Gln Met Asp Val Asn Pro Glu Gly Lys Tyr Ser Phe Gly
                275 280 285
Ala Thr Cys Val Lys Lys Cys Pro Arg Asn Tyr Val Val Thr Asp His
        290 295 300
Gly Ser Cys Val Arg Ala Cys Gly Ala Asp Ser Tyr Glu Met Glu Glu
305 310 315 320
Asp Gly Val Arg Lys Cys Lys Lys Cys Glu Gly Pro Cys Arg Lys Val
                                325 330 335
Cys Asn Gly Ile Gly Ile Gly Glu Phe Lys Asp Ser Leu Ser Ile Asn
                        340 345 350
Ala Thr Asn Ile Lys His Phe Lys Asn Cys Thr Ser Ile Ser Gly Asp
                355 360 365
Leu His Ile Leu Pro Val Ala Phe Arg Gly Asp Ser Phe Thr His Thr
        370 375 380
Pro Pro Leu Asp Pro Gln Glu Leu Asp Ile Leu Lys Thr Val Lys Glu
385 390 395 400
Ile Thr Gly Phe Leu Leu Ile Gln Ala Trp Pro Glu Asn Arg Thr Asp
                                405 410 415
Leu His Ala Phe Glu Asn Leu Glu Ile Ile Arg Gly Arg Thr Lys Gln
                        420 425 430
His Gly Gln Phe Ser Leu Ala Val Val Ser Leu Asn Ile Thr Ser Leu
                435 440 445
Gly Leu Arg Ser Leu Lys Glu Ile Ser Asp Gly Asp Val Ile Ile Ser
        450 455 460
Gly Asn Lys Asn Leu Cys Tyr Ala Asn Thr Ile Asn Trp Lys Lys Leu
465 470 475 480
Phe Gly Thr Ser Gly Gln Lys Thr Lys Ile Ile Ser Asn Arg Gly Glu
                                485 490 495
Asn Ser Cys Lys Ala Thr Gly Gln Val Cys His Ala Leu Cys Ser Pro
                        500 505 510
Glu Gly Cys Trp Gly Pro Glu Pro Arg Asp Cys Val Ser Cys Arg Asn
                515 520 525
Val Ser Arg Gly Arg Glu Cys Val Asp Lys Cys Asn Leu Leu Glu Gly
        530 535 540
Glu Pro Arg Glu Phe Val Glu Asn Ser Glu Cys Ile Gln Cys His Pro
545 550 555 560
Glu Cys Leu Pro Gln Ala Met Asn Ile Thr Cys Thr Gly Arg Gly Pro
                                565 570 575
Asp Asn Cys Ile Gln Cys Ala His Tyr Ile Asp Gly Pro His Cys Val
                        580 585 590
Lys Thr Cys Pro Ala Gly Val Met Gly Glu Asn Asn Thr Leu Val Trp
                595 600 605
Lys Tyr Ala Asp Ala Gly His Val Cys His Leu Cys His Pro Asn Cys
        610 615 620
Thr Tyr Gly Cys Thr Gly Pro Gly Leu Glu Gly Cys Pro Thr Asn Gly
625 630 635 640
Pro Lys Ile Pro Ser Ile Ala Thr Gly Met Val Gly Ala Leu Leu Leu
                                645 650 655
Leu Leu Val Val Ala Leu Gly Ile Gly Leu Phe Met Arg Arg Arg His
                        660 665 670
Ile Val Arg Lys Arg Thr Leu Arg Arg Leu Leu Gln Glu Arg Glu Leu
                675 680 685
Val Glu Pro Leu Thr Pro Ser Gly Glu Ala Pro Asn Gln Ala Leu Leu
        690 695 700
Arg Ile Leu Lys Glu Thr Glu Phe Lys Lys Ile Lys Val Leu Gly Ser
705 710 715 720
Gly Ala Phe Gly Thr Val Tyr Lys Gly Leu Trp Ile Pro Glu Gly Glu
                                725 730 735
Lys Val Lys Ile Pro Val Ala Ile Lys Glu Leu Arg Glu Ala Thr Ser
                        740 745 750
Pro Lys Ala Asn Lys Glu Ile Leu Asp Glu Ala Tyr Val Met Ala Ser
                755 760 765
Val Asp Asn Pro His Val Cys Arg Leu Leu Gly Ile Cys Leu Thr Ser
        770 775 780
Thr Val Gln Leu Ile Thr Gln Leu Met Pro Phe Gly Cys Leu Leu Asp
785 790 795 800
Tyr Val Arg Glu His Lys Asp Asn Ile Gly Ser Gln Tyr Leu Leu Asn
                                805 810 815
Trp Cys Val Gln Ile Ala Lys Gly Met Asn Tyr Leu Glu Asp Arg Arg
                        820 825 830
Leu Val His Arg Asp Leu Ala Ala Arg Asn Val Leu Val Lys Thr Pro
                835 840 845
Gln His Val Lys Ile Thr Asp Phe Gly Leu Ala Lys Leu Leu Gly Ala
        850 855 860
Glu Glu Lys Glu Tyr His Ala Glu Gly Gly Lys Val Pro Ile Lys Trp
865 870 875 880
Met Ala Leu Glu Ser Ile Leu His Arg Ile Tyr Thr His Gln Ser Asp
                                885 890 895
Val Trp Ser Tyr Gly Val Thr Val Trp Glu Leu Met Thr Phe Gly Ser
                        900 905 910
Lys Pro Tyr Asp Gly Ile Pro Ala Ser Glu Ile Ser Ser Ile Leu Glu
                915 920 925
Lys Gly Glu Arg Leu Pro Gln Pro Pro Ile Cys Thr Ile Asp Val Tyr
        930 935 940
Met Ile Met Val Lys Cys Trp Met Ile Asp Ala Asp Ser Arg Pro Lys
945 950 955 960
Phe Arg Glu Leu Ile Ile Glu Phe Ser Lys Met Ala Arg Asp Pro Gln
                                965 970 975
Arg Tyr Leu Val Ile Gln Gly Asp Glu Arg Met His Leu Pro Ser Pro
                        980 985 990
Thr Asp Ser Asn Phe Tyr Arg Ala Leu Met Asp Glu Glu Asp Met Asp
                995 1000 1005
Asp Val Val Asp Ala Asp Glu Tyr Leu Ile Pro Gln Gln Gly Phe
        1010 1015 1020
Phe Ser Ser Pro Ser Thr Ser Arg Thr Pro Leu Leu Ser Ser Leu
        1025 1030 1035
Ser Ala Thr Ser Asn Asn Ser Thr Val Ala Cys Ile Asp Arg Asn
        1040 1045 1050
Gly Leu Gln Ser Cys Pro Ile Lys Glu Asp Ser Phe Leu Gln Arg
        1055 1060 1065
Tyr Ser Ser Asp Pro Thr Gly Ala Leu Thr Glu Asp Ser Ile Asp
        1070 1075 1080
Asp Thr Phe Leu Pro Val Pro Glu Tyr Ile Asn Gln Ser Val Pro
        1085 1090 1095
Lys Arg Pro Ala Gly Ser Val Gln Asn Pro Val Tyr His Asn Gln
        1100 1105 1110
Pro Leu Asn Pro Ala Pro Ser Arg Asp Pro His Tyr Gln Asp Pro
        1115 1120 1125
His Ser Thr Ala Val Gly Asn Pro Glu Tyr Leu Asn Thr Val Gln
        1130 1135 1140
Pro Thr Cys Val Asn Ser Thr Phe Asp Ser Pro Ala His Trp Ala
        1145 1150 1155
Gln Lys Gly Ser His Gln Ile Ser Leu Asp Asn Pro Asp Tyr Gln
        1160 1165 1170
Gln Asp Phe Phe Pro Lys Glu Ala Lys Pro Asn Gly Ile Phe Lys
        1175 1180 1185
Gly Ser Thr Ala Glu Asn Ala Glu Tyr Leu Arg Val Ala Pro Gln
        1190 1195 1200
Ser Ser Glu Phe Ile Gly Ala
        1205 1210
```

| | |
|---|---|
| <210> | 9 |
| <211> | 142 |
| <212> | PRT |
| <213> | Homo sapiens |
| <400> | 9 |

```
Met Gly Ala Pro Thr Leu Pro Pro Ala Trp Gln Pro Phe Leu Lys Asp
1 5 10 15
His Arg Ile Ser Thr Phe Lys Asn Trp Pro Phe Leu Glu Gly Cys Ala
                        20 25 30
Cys Thr Pro Glu Arg Met Ala Glu Ala Gly Phe Ile His Cys Pro Thr
                35 40 45
Glu Asn Glu Pro Asp Leu Ala Gln Cys Phe Phe Cys Phe Lys Glu Leu
        50 55 60
Glu Gly Trp Glu Pro Asp Asp Asp Pro Ile Glu Glu His Lys Lys His
65 70 75 80
Ser Ser Gly Cys Ala Phe Leu Ser Val Lys Lys Gln Phe Glu Glu Leu
                                85 90 95
Thr Leu Gly Glu Phe Leu Lys Leu Asp Arg Glu Arg Ala Lys Asn Lys
                        100 105 110
Ile Ala Lys Glu Thr Asn Asn Lys Lys Lys Glu Phe Glu Glu Thr Ala
                115 120 125
Lys Lys Val Arg Arg Ala Ile Glu Gln Leu Ala Ala Met Asp
        130 135 140
```

| | |
|---|---|
| <210> | 10 |
| <211> | 435 |
| <212> | PRT |
| <213> | Homo sapiens |
| <400> | 10 |

```
Met Glu Met Glu Lys Glu Phe Glu Gln Ile Asp Lys Ser Gly Ser Trp
1 5 10 15
Ala Ala Ile Tyr Gln Asp Ile Arg His Glu Ala Ser Asp Phe Pro Cys
                        20 25 30
Arg Val Ala Lys Leu Pro Lys Asn Lys Asn Arg Asn Arg Tyr Arg Asp
                35 40 45
Val Ser Pro Phe Asp His Ser Arg Ile Lys Leu His Gln Glu Asp Asn
        50 55 60
Asp Tyr Ile Asn Ala Ser Leu Ile Lys Met Glu Glu Ala Gln Arg Ser
65 70 75 80
Tyr Ile Leu Thr Gln Gly Pro Leu Pro Asn Thr Cys Gly His Phe Trp
                                85 90 95
Glu Met Val Trp Glu Gln Lys Ser Arg Gly Val Val Met Leu Asn Arg
                        100 105 110
Val Met Glu Lys Gly Ser Leu Lys Cys Ala Gln Tyr Trp Pro Gln Lys
                115 120 125
Glu Glu Lys Glu Met Ile Phe Glu Asp Thr Asn Leu Lys Leu Thr Leu
        130 135 140
Ile Ser Glu Asp Ile Lys Ser Tyr Tyr Thr Val Arg Gln Leu Glu Leu
145 150 155 160
Glu Asn Leu Thr Thr Gln Glu Thr Arg Glu Ile Leu His Phe His Tyr
                                165 170 175
Thr Thr Trp Pro Asp Phe Gly Val Pro Glu Ser Pro Ala Ser Phe Leu
                        180 185 190
Asn Phe Leu Phe Lys Val Arg Glu Ser Gly Ser Leu Ser Pro Glu His
                195 200 205
Gly Pro Val Val Val His Cys Ser Ada Gly Ile Gly Arg Ser Gly Thr
        210 215 220
Phe Cys Leu Ala Asp Thr Cys Leu Leu Leu Met Asp Lys Arg Lys Asp
225 230 235 240
Pro Ser Ser Val Asp Ile Lys Lys Val Leu Leu Glu Met Arg Lys Phe
                                245 250 255
Arg Met Gly Leu Ile Gln Thr Ala Asp Gln Leu Arg Phe Ser Tyr Leu
                        260 265 270
Ala Val Ile Glu Gly Ala Lys Phe Ile Met Gly Asp Ser Ser Val Gln
                275 280 285
Asp Gln Trp Lys Glu Leu Ser His Glu Asp Leu Glu Pro Pro Pro Glu
        290 295 300
His Ile Pro Pro Pro Pro Arg Pro Pro Lys Arg Ile Leu Glu Pro His
305 310 315 320
Asn Gly Lys Cys Arg Glu Phe Phe Pro Asn His Gln Trp Val Lys Glu
                                325 330 335
Glu Thr Gln Glu Asp Lys Asp Cys Pro Ile Lys Glu Glu Lys Gly Ser
                        340 345 350
Pro Leu Asn Ala Ala Pro Tyr Gly Ile Glu Ser Met Ser Gln Asp Thr
                355 360 365
Glu Val Arg Ser Arg Val Val Gly Gly Ser Leu Arg Gly Ala Gln Ala
        370 375 380
Ala Ser Pro Ala Lys Gly Glu Pro Ser Leu Pro Glu Lys Asp Glu Asp
385 390 395 400
His Ala Leu Ser Tyr Trp Lys Pro Phe Leu Val Asn Met Cys Val Ala
                                405 410 415
Thr Val Leu Thr Ala Gly Ala Tyr Leu Cys Tyr Arg Phe Leu Phe Asn
                        420 425 430
Ser Asn Thr
                435
```

| | |
|---|---|
| <210> | 11 |
| <211> | 232 |
| <212> | PRT |
| <213> | Homo sapiens |
| <400> | 11 |

```
Met Asn Phe Leu Leu Ser Trp Val His Trp Ser Leu Ala Leu Leu Leu
1 5 10 15
Tyr Leu His His Ala Lys Trp Ser Gln Ala Ala Pro Met Ala Glu Gly
                        20 25 30
Gly Gly Gln Asn His His Glu Val Val Lys Phe Met Asp Val Tyr Gln
                35 40 45
Arg Ser Tyr Cys His Pro Ile Glu Thr Leu Val Asp Ile Phe Gln Glu
        50 55 60
Tyr Pro Asp Glu Ile Glu Tyr Ile Phe Lys Pro Ser Cys Val Pro Leu
65 70 75 80
Met Arg Cys Gly Gly Cys Cys Asn Asp Glu Gly Leu Glu Cys Val Pro
                                85 90 95
Thr Glu Glu Ser Asn Ile Thr Met Gln Ile Met Arg Ile Lys Pro His
                        100 105 110
Gln Gly Gln His Ile Gly Glu Met Ser Phe Leu Gln His Asn Lys Cys
                115 120 125
Glu Cys Arg Pro Lys Lys Asp Arg Ala Arg Gln Glu Lys Lys Ser Val
        130 135 140
Arg Gly Lys Gly Lys Gly Gln Lys Arg Lys Arg Lys Lys Ser Arg Tyr
145 150 155 160
Lys Ser Trp Ser Val Tyr Val Gly Ala Arg Cys Cys Leu Met Pro Trp
                                165 170 175
Ser Leu Pro Gly Pro His Pro Cys Gly Pro Cys Ser Glu Arg Arg Lys
                        180 185 190
His Leu Phe Val Gln Asp Pro Gln Thr Cys Lys Cys Ser Cys Lys Asn
                195 200 205
Thr Asp Ser Arg Cys Lys Ala Arg Gln Leu Glu Leu Asn Glu Arg Thr
        210 215 220
Cys Arg Cys Asp Lys Pro Arg Arg
225 230
```

| | |
|---|---|
| <210> | 12 |
| <211> | 889 |
| <212> | PRT |
| <213> | Homo sapiens |
| <400> | 12 |

```
Met Glu Glu Gly Ala Pro Arg Gln Pro Gly Pro Ser Gln Trp Pro Pro
1 5 10 15
Glu Asp Glu Lys Glu Val Ile Arg Arg Ala Ile Gln Lys Glu Leu Lys
                        20 25 30
Ile Lys Glu Gly Val Glu Asn Leu Arg Arg Val Ala Thr Asp Arg Arg
                35 40 45
His Leu Gly His Val Gln Gln Leu Leu Arg Ser Ser Asn Arg Arg Leu
        50 55 60
Glu Gln Leu His Gly Glu Leu Arg Glu Leu His Ala Arg Ile Leu Leu
65 70 75 80
Pro Gly Pro Gly Pro Gly Pro Ala Glu Pro Val Ala Ser Gly Pro Arg
                                85 90 95
Pro Trp Ala Glu Gln Leu Arg Ala Arg His Leu Glu Ala Leu Arg Arg
                        100 105 110
Gln Leu His Val Glu Leu Lys Val Lys Gln Gly Ala Glu Asn Met Thr
                115 120 125
His Thr Cys Ala Ser Gly Thr Pro Lys Glu Arg Lys Leu Leu Ala Ala
        130 135 140
Ala Gln Gln Met Leu Arg Asp Ser Gln Leu Lys Val Ala Leu Leu Arg
145 150 155 160
Met Lys Ile Ser Ser Leu Glu Ala Ser Gly Ser Pro Glu Pro Gly Pro
                                165 170 175
Glu Leu Leu Ala Glu Glu Leu Gln His Arg Leu His Val Glu Ala Ala
                        180 185 190
Val Ala Glu Gly Ala Lys Asn Val Val Lys Leu Leu Ser Ser Arg Arg
                195 200 205
Thr Gln Asp Arg Lys Ala Leu Ala Glu Ala Gln Ala Gln Leu Gln Glu
        210 215 220
Ser Ser Gln Lys Leu Asp Leu Leu Arg Leu Ala Leu Glu Gln Leu Leu
225 230 235 240
Glu Gln Leu Pro Pro Ala His Pro Leu Arg Ser Arg Val Thr Arg Glu
                                245 250 255
Leu Arg Ala Ala Val Pro Gly Tyr Pro Gln Pro Ser Gly Thr Pro Val
                        260 265 270
Lys Pro Thr Ala Leu Thr Gly Thr Leu Gln Val Arg Leu Leu Gly Cys
                275 280 285
Glu Gln Leu Leu Thr Ala Val Pro Gly Arg Ser Pro Ala Ala Ala Leu
        290 295 300
Ala Ser Ser Pro Ser Glu Gly Trp Leu Arg Thr Lys Ala Lys His Gln
305 310 315 320
Arg Gly Arg Gly Glu Leu Ala Ser Glu Val Leu Ala Val Leu Lys Val
                                325 330 335
Asp Asn Arg Val Val Gly Gln Thr Gly Trp Gly Gln Val Ala Glu Gln
                        340 345 350
Ser Trp Asp Gln Thr Phe Val Ile Pro Leu Glu Arg Ala Arg Glu Leu
                355 360 365
Glu Ile Gly Val His Trp Arg Asp Trp Arg Gln Leu Cys Gly Val Ala
        370 375 380
Phe Leu Arg Leu Glu Asp Phe Leu Asp Asn Ala Cys His Gln Leu Ser
385 390 395 400
Leu Ser Leu Val Pro Gln Gly Leu Leu Phe Ala Gln Val Thr Phe Cys
                                405 410 415
Asp Pro Val Ile Glu Arg Arg Pro Arg Leu Gln Arg Gln Glu Arg Ile
                        420 425 430
Phe Ser Lys Arg Arg Gly Gln Asp Phe Leu Arg Ala Ser Gln Met Asn
                435 440 445
Leu Gly Met Ala Ala Trp Gly Arg Leu Val Met Asn Leu Leu Pro Pro
        450 455 460
Cys Ser Ser Pro Ser Thr Ile Ser Pro Pro Lys Gly Cys Pro Arg Thr
465 470 475 480
Pro Thr Thr Leu Arg Glu Ala Ser Asp Pro Ala Thr Pro Ser Asn Phe
                                485 490 495
Leu Pro Lys Lys Thr Pro Leu Gly Glu Glu Met Thr Pro Pro Pro Lys
                        500 505 510
Pro Pro Arg Leu Tyr Leu Pro Gln Glu Pro Thr Ser Glu Glu Thr Pro
                515 520 525
Arg Thr Lys Arg Pro His Met Glu Pro Arg Thr Arg Arg Gly Pro Ser
        530 535 540
Pro Pro Ala Ser Pro Thr Arg Lys Pro Pro Arg Leu Gln Asp Phe Arg
545 550 555 560
Cys Leu Ala Val Leu Gly Arg Gly His Phe Gly Lys Val Leu Leu Val
                                565 570 575
Gln Phe Lys Gly Thr Gly Lys Tyr Tyr Ala Ile Lys Ala Leu Lys Lys
                        580 585 590
Gln Glu Val Leu Ser Arg Asp Glu Ile Glu Ser Leu Tyr Cys Glu Lys
                595 600 605
Arg Ile Leu Glu Ala Val Gly Cys Thr Gly His Pro Phe Leu Leu Ser
        610 615 620
Leu Leu Ala Cys Phe Gln Thr Ser Ser His Ala Cys Phe Val Thr Glu
625 630 635 640
Phe Val Pro Gly Gly Asp Leu Met Met Gln Ile His Glu Asp Val Phe
                                645 650 655
Pro Glu Pro Gln Ala Arg Phe Tyr Val Ala Cys Val Val Leu Gly Leu
                        660 665 670
Gln Phe Leu His Glu Lys Lys Ile Ile Tyr Arg Asp Leu Lys Leu Asp
                675 680 685
Asn Leu Leu Leu Asp Ala Gln Gly Phe Leu Lys Ile Ala Asp Phe Gly
        690 695 700
Leu Cys Lys Glu Gly Ile Gly Phe Gly Asp Arg Thr Ser Thr Phe Cys
705 710 715 720
Gly Thr Pro Glu Phe Leu Ala Pro Glu Val Leu Thr Gln Glu Ala Tyr
                                725 730 735
Thr Arg Ala Val Asp Trp Trp Gly Leu Gly Val Leu Leu Tyr Glu Met
                        740 745 750
Leu Val Gly Glu Cys Pro Phe Pro Gly Asp Thr Glu Glu Glu Val Phe
                755 760 765
Asp Cys Ile Val Asn Met Asp Ala Pro Tyr Pro Gly Phe Leu Ser Val
        770 775 780
Gln Gly Leu Glu Phe Ile Gln Lys Leu Leu Gln Lys Cys Pro Glu Lys
785 790 795 800
Arg Leu Gly Ala Gly Glu Gln Asp Ala Glu Glu Ile Lys Val Gln Pro
                                805 810 815
Phe Phe Arg Thr Thr Asn Trp Gln Ala Leu Leu Ala Arg Thr Ile Gln
                        820 825 830
Pro Pro Phe Val Pro Thr Leu Cys Gly Pro Ala Asp Leu Arg Tyr Phe
                835 840 845
Glu Gly Glu Phe Thr Gly Leu Pro Pro Ala Leu Thr Pro Pro Ala Pro
        850 855 860
His Ser Leu Leu Thr Ala Arg Gln Gln Ala Ala Phe Arg Asp Phe Asp
865 870 875 880
Phe Val Ser Glu Arg Phe Leu Glu Pro
                                885
```

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for creating an intracellular antibody delivery device comprising:
forming a polymer for introduction to a cell;
producing a polymeric nanogel via crosslinking;
introducing an antibody or protein to the polymeric nanogel, wherein the antibody or protein is internalized by the nanogel;
uptake by at least one cell of the polymeric nanogel; and
cleaving a self-immolative linker present in the polymeric nanogel to release the antibody or protein within the at least one cell.

2. The method of claim 1, wherein the polymer comprises PDA-PEG-NPC.

3. The method of claim 1, wherein the polymer comprises PDA-PEG-BSA-Cy5 or PDA-PEG-Cy3.

4. The method of 2, wherein the PDA-PEG-NPC polymer comprises p-nitrophenylcarbonate (NPC) moieties in side chains.

5. The method of 4, wherein the NPC moieties are replaced by lysine groups of the antibody or protein to produce antibody or protein conjugated polymers.

6. The method of claim 1, wherein the nanogel comprises PBS buffer, TCEP and ethylenediamine and deionized water.

7. The method of claim 1, wherein the nanogel is modified with RGD peptide.

8. The method of claim 1, wherein the at least one cell is a human breast cancer cell.

9. The method of claim 8, wherein the method degrades a protein containing SEQ ID NO: 1, SEQ ID NO: 2, and/or SEQ ID NO: 3 in the at least one human breast cancer cell.

10. The method of claim 1, wherein the method is employed to treat cancer, Alzheimer's diseases, Parkinson's disease, multiple sclerosis, neonatal hypoxic-ischemic, stroke, Amyotrophic lateral sclerosis, Huntington's disease, spinal cord injury, brain injury, retina injury, post-traumatic stress disorder, and frontotemporal dementia, and/or traumatic brain injury.

11. A method for degrading intracellular proteins in at least one TRIM21 expressing cell comprising:
forming at least one protein loaded nanogel wherein the protein loaded nanogel comprises at least one polymer nanogel and at least one protein; and
wherein the at least one protein comprises at least one antibody, at least one nanobody, or a combinations of at least one antibody and at least one nanobody.

12. The method of claim 11, wherein the TRIM21 expressing cell is a naturally occurring TRIM21 expressing cell or a cell with acquired TRIM21 expression.

13. The method of claim 11, wherein the at least one antibody comprises anti COPZ1 antibody, anti PTBP1 antibody, anti PD-L1 antibody, anti PD-1 antibody, anti-Her2 antibody, anti EGFR antibody, anti survivin antibody, anti PTP1B antibody, anti VEGF antibody, anti PKN3 antibody.

14. The method of claim 11, wherein the at least one nanobody comprises anti COPZ1 nanobody, anti PTBP1 nanobody, anti PD-L1 nanobody, anti PD-1 nanobody, anti-Her2 nanobody, anti EGFR nanobody, anti survivin nanobody, anti PTP1B nanobody, anti VEGF nanobody, anti PKN3 nanobody.

15. The method of claim 11, wherein the protein-laded nanogel system is used for treating cancer, Alzheimer's diseases, Parkinson's disease, multiple sclerosis, neonatal hypoxic-ischemic, stroke, Amyotrophic lateral sclerosis, Huntington's disease, spinal cord injury, brain injury, retina injury, post-traumatic stress disorder, and frontotemporal dementia, and/or traumatic brain injury.
